(19) United States
(12) Patent Application Publication
(10) Pub. No.: US 2010/0068766 A1
(43) Pub. Date: Mar. 18, 2010

(54) COMPOSITION FOR CLEAVING AND/OR CONNECTING SINGLE STRAND DNA

(75) Inventors: Hitoshi Kurumizaka, Tokyo (JP); Motoki Takaku, Tokyo (JP); Shinichi Machida, Tokyo (JP)

Correspondence Address: PEARNE & GORDON LLP, 1801 EAST 9TH STREET, SUITE 1200, CLEVELAND, OH, 44114-3108, US

(73) Assignee: WASEDA UNIVERSITY, Tokyo (JP)

(21) Appl. No.: 12/529,616

(22) PCT Filed: Mar. 7, 2008
(86) PCT No.: PCT/JP2008/000477

§ 371 Date: Sep. 2, 2009

(30) Foreign Application Priority Data
Mar. 7, 2007 (JP) 2007-056550

(51) Int. Cl. C12P 19/34; C12N 9/90; C07H 21/04; C12N 9/99

(52) U.S. Cl. 435/91.5; 435/233; 536/23.1; 435/184

(57) ABSTRACT

It is an object of the present invention to provide a composition for catalyzing the cleavage of a single-stranded DNA and the binding of such single-stranded DNA. The present invention provides a composition for cleaving a single-stranded DNA and/or binding the 5′-terminus of such single-stranded DNA to the 3′-terminus thereof, which comprises an Ev1 protein. Moreover, the present invention also provides a composition for cleaving a single-stranded DNA and/or binding the 5′-terminus of such single-stranded DNA to the 3′-terminus thereof, which further comprises a Rad51B protein and/or a DNA topoisomerase type I protein, as well as the Ev1 protein.

### TECHNICAL FIELD

The present invention relates to a composition for cleaving and/or binding single-stranded DNA.

### BACKGROUND ART

Recombinant DNA technology, which makes full use of genetic engineering techniques, is an important technology that is essential for the development of the current biotechnology industry. For example, in recent years, in the field of medicine, it is almost impossible to supply pharmaceutical products to meet demand in the development and production of protein preparations whose efficacy has been focused, without using such recombinant DNA technology. Thus, it may be no exaggeration to say that the development of the current medical field cannot be anticipated without this technology. In addition, it is desired to further develop DNA manipulation technology, not only for the development of protein preparations but also for the development of protein reagents used in research and development.

The most basic technique for dealing with DNA recombination operations includes the cleavage of DNA and the binding of a free terminus. A majority of DNA molecules used as targets of such operations have been double-stranded DNA molecules. To date, since DNA has been often used as a “tool for encoding a protein and expressing it,” most researchers have been interested in the progress of a technique of manipulating a double-stranded DNA. However, at present, with the expansion of a biotechnology target region, opportunities for manipulating a single-stranded DNA have been increased. For example, when a DNA chip or the like is produced, it is essential to prepare a single-stranded DNA having a desired sequence and a desired length, and further, an enzyme and the like used to manipulate such single-stranded DNA are also considered as important factors. A large number of methods for cleaving a single-stranded DNA have been reported so far. However, as methods for binding a single-stranded DNA to another single-stranded DNA, there have been reported only several methods such as a method using T4 RNA ligase (Patent Document 1 and Non-Patent Document 1), a method using thermostable ligase derived from archaebacteria (Patent Document 2), and a method using thermostable ligase derived from thermophilic phage TS2126 (Patent Document 3).

Under such circumstances, it is desired to develop an enzyme or a composition capable of manipulating available single-stranded DNA molecules.
[Patent Document 1] JP Patent Publication (Kokai) No. 2002-171983 A (the entire text)
[Patent Document 2] JP Patent Publication (Kokai) No. 6-62847 A (1994) (the entire text)
[Patent Document 3] U.S. Pat. No. 6,818,425 (the entire text)
[Non-Patent Document 1] Nishigaki et al., Mol Divers 4: 187-90, 1998

### DISCLOSURE OF THE INVENTION

### Problems to be Solved by the Invention

Under the aforementioned circumstances, the present inventors have conducted intensive studies. As a result, the inventors have found that an Ev1 protein has activity of cleaving a single-stranded DNA and binding the 5′-terminus of such single-stranded DNA to the 3′-terminus thereof, thereby completing the present invention.

Accordingly, it is an object of the present invention to provide a composition for cleaving and/or binding a single-stranded DNA.

### Means for Solving the Problems

Specifically, the present invention relates to the following (1) to (12):

(1) A first aspect of the present invention relates to “a composition for cleaving a single-stranded DNA and/or binding the 5′-terminus of such single-stranded DNA to the 3′-terminus thereof, which comprises an Ev1 protein described in the following (a) or (b):
(a) a protein having the amino acid sequence shown in SEQ ID NO: 2, 4, 6, 8, 10, 12, or 20; or
(b) a polypeptide, which has an amino acid sequence comprising a substitution, deletion, or insertion of one or several amino acids with respect to the amino acid sequence shown in SEQ ID NO: 2, 4, 6, 8, 10, 12, or 20, and which has activity of cleaving a single-stranded DNA and binding the 5′-terminus of such single-stranded DNA to the 3′-terminus thereof.”
(2) A second aspect of the present invention relates to “the composition according to (1) above, wherein it further comprises Mg²⁺ or Ca²⁺.”
(3) A third aspect of the present invention relates to “the composition according to (2) above, wherein the concentration of the Mg²⁺ is between 0.5 mM and 2.0 mM.”
(4) A fourth aspect of the present invention relates to “the composition according to (3) above, wherein it further comprises a Rad51B protein and ATP.”
(5) A fifth aspect of the present invention relates to “the composition according to (4) above, wherein the molar ratio of the Ev1 protein to the Rad51B is from 1:0.5 to 1:4.”
(6) A sixth aspect of the present invention relates to “the composition according to (4) or (5) above, wherein the concentration of the ATP is between 0.5 mM and 2.0 mM.”
(7) A seventh aspect of the present invention relates to “the composition according to any one of (1) to (3) above, wherein it further comprises a DNA topoisomerase type I protein.”
(8) An eighth aspect of the present invention relates to “a composition for cleaving a single-stranded DNA and/or binding the 5′-terminus of such single-stranded DNA to the 3′-terminus thereof, which comprises a recombinant vector comprising a nucleic acid described in the following (a) or (b):
(a) a nucleic acid having the nucleotide sequence shown in SEQ ID NO: 1, 3, 5, 7, 9, 11, or 19; or
(b) a nucleic acid, which hybridizes under stringent conditions with the complementary strand of the nucleic acid having the nucleotide sequence shown in SEQ ID NO: 1, 3, 5, 7, 9, 11, or 19, and which encodes a polypeptide having activity of cleaving a single-stranded DNA and binding the 5′-terminus of such single-stranded DNA to the 3′-terminus thereof.”
(9) A ninth aspect of the present invention relates to “a composition for cleaving a single-stranded DNA and/or binding the 5′-terminus of such single-stranded DNA to the 3′-terminus thereof, which comprises a recombinant vector comprising a nucleic acid described in the following (a) or (b):
(a) a nucleic acid encoding a polypeptide having the amino acid sequence shown in SEQ ID NO: 2, 4, 6, 8, 10, 12, or 20; or
(b) a nucleic acid encoding a polypeptide, which has an amino acid sequence comprising a substitution, deletion, or insertion of one or several amino acids with respect to the amino acid sequence shown in SEQ ID NO: 2, 4, 6, 8, 10, 12, or 20, and which has activity of cleaving a single-stranded DNA and binding the 5′-terminus of such single-stranded DNA to the 3′-terminus thereof.”
(10) A tenth aspect of the present invention relates to “a method for producing a single-stranded DNA marker by reacting the composition according to any one of (1) to (9) above with a single-stranded DNA.”
(11) An eleventh aspect of the present invention relates to “a single-stranded DNA marker produced by the method according to (10) above.”
(12) A twelfth aspect of the present invention relates to “a composition for inhibiting Ev1 protein activity, which comprises one or multiple compounds selected from the compound group consisting of aclarubicin, dequalinium, DIDS, β-rubromycin, and 3-ATA.”

### EFFECTS OF THE INVENTION

According to the method of the present invention, a single-stranded DNA can be cleaved, and/or the 5′-terminus of such single-stranded DNA can be bound to the 3′-terminus thereof.

### BRIEF DESCRIPTION OF THE DRAWINGS

FIG. 1 shows the results indicating that an Ev1 protein binds to a Rad51B protein. In FIG. 1a, the purified Ev1 protein (0.5 μg) and Rad51B protein (0.5 μg) were electrophoresed by 12% SDS-PAGE, and were then strained with Coomassie Brilliant Blue. Lane 1 indicates markers, and lanes 2 and 3 are the purified Ev1 and the purified Rad51B, respectively. FIG. 1b shows the results indicating the interaction of Ev1-Rad51B. Lanes 1 and 2 indicate the results of a binding experiment using Ev1-bound beads in the absence (lane 1) or presence (lane 2) of Rad51B. Lane 3 indicates the experimental results of a negative control, in which Affi-Gel beads to which no protein had bound were used. FIG. 1c shows the results of the interaction of Ev1-Rad51B. Lanes 1 and 2 indicate the results of a binding experiment using Rad51B-bound beads in the absence (lane 1) or presence (lane 2) of Ev1. Lane 3 indicates the experimental results of a negative control, in which Affi-Gel beads to which no protein had bound were used.

FIG. 2 shows the results obtained by gel filtration of the purified Ev1 protein. The Ev1 protein was eluted at a position of molecular weight of approximately 600 kDa.

FIG. 3 shows the results obtained by examining the DNA binding ability of the Ev1 protein. Lanes 1-6 and lanes 7-12 indicate the results obtained by examining the ability of the Ev1 protein to bind to a double-stranded DNA and to a single-stranded DNA, respectively. Lanes 1 and 7 indicate the results of a negative control, to which no Ev1 protein was added. The concentrations of Ev1 proteins used in such binding experiments are 0.2 μM (lanes 2 and 8), 0.4 μM (lanes 3 and 9), 0.8 μM (lanes 4 and 10), 1.6 μM (lanes 5 and 11), and 3.2 μM (lanes 6 and 12). The symbol “nc” indicates a nicked circular double-stranded DNA, the symbol “sc” indicates a supercoiled circular double-stranded DNA, and the symbol “ss” indicates a single-stranded DNA (this also applies to other figures).

FIG. 4 shows the results obtained by examining the activity of cleaving a single-stranded DNA and binding the 5′-terminus of such single-stranded DNA to the 3′-terminus thereof. FIG. 4a shows the results obtained by examining the activity of Ev1 to a circular single-stranded DNA. Lanes 1 and 2 indicate the results obtained using a φ×174 circular single-stranded DNA. Lanes 3 and 4 indicate the results obtained using a M13 mp18 circular single-stranded DNA. In addition, lanes 1 and 3 indicate the results of control experiments in which no Ev1 protein was used, and lanes 2 and 4 indicate the results of experiments in which the Ev1 protein was used. FIG. 4b shows the results obtained by examining the activity of Ev1 to double-stranded, supercoiled DNA and nicked DNA. A φ×174 circular single-stranded DNA (20 μM, lanes 1-3), or φ×174 supercoiled and nicked double-stranded DNA molecules (10 μM, lanes 4-6) were used to examine the influence of Ev1 (4 μM, lanes 2 and 5) and Escherichia coli-derived topoisomerase type I (5 units, lanes 3 and 6). Lanes 1 and 4 indicate the results of control experiments in which no Ev1 protein was used.

FIG. 5 shows the influence of Mg²⁺ and divalent metal ions on the activity of an Ev1 protein. FIG. 5a shows the results obtained by examining the influence of Mg²⁺ on the activity of the Ev1 protein. A catenation reaction was carried out on a φ×174 circular single-stranded DNA (20 μM) using an Ev1 protein (4 μM) in the presence of various concentrations of Mg²⁺. Lane 1 indicates the results of control experiments in which no Ev1 protein was used. In addition, lanes 2-8 indicate experimental results in the presence of Mg²⁺ in concentrations of 0 mM, 0.5 mM, 1.0 mM, 1.25 mM, 1.5 mM, 1.75 mM, and 2.0 mM, respectively. FIG. 5b shows the results obtained by examining the influence of divalent metal ions on the activity of an Ev1 protein. A catenation reaction was carried out on a φ×174 circular single-stranded DNA (20 μM) using an Ev1 protein (4 μM) in the presence of various types of divalent metal ions. Lane 1 indicates the results of a control experiment in which no metal ion was used. In addition, lanes 2-5 indicate experimental results in the presence of 1 mM metal ions of several types as shown in the figure.

FIG. 6 shows the results obtained by examining the heat stability of a reaction product obtained using an Ev1 protein. The lanes show the results obtained by incubating at 100° C. a reaction product obtained by reacting a φ×174 circular single-stranded DNA (20 μM) with an Ev1 protein (4 μM) for 0 minute (lane 2), 0.5 minutes (lane 3), 1 minute (lane 4), 5 minutes (lane 5), and 10 minutes (lane 6), followed by agarose gel electrophoresis. Lane 1 indicates the results of a control experiment in which no Ev1 protein was used.

FIG. 7 shows the results obtained by observing a reaction product obtained using an Ev1 protein under an electron microscope. The scale bar indicates 100 nm.

FIG. 8 shows the results indicating that a Rad51B protein promotes the activity of an Ev1 protein. FIG. 8a shows the results obtained by examining the influence of the Rad51B protein on the activity of the Ev1 protein. Lanes 1 and 5 indicate the results of control experiments in which neither the Ev1 nor the Rad51B protein was used. Lanes 2 and 6 indicate the results of experiments that were carried out using a 4 μM Rad51B protein in the absence of the Ev1 protein. Lanes 3 and 7 indicate the results of experiments that were carried out using a 4 μM Ev1 protein in the absence of the Rad51B protein. Lanes 4 and 8 indicate the results of experiments that were carried out using a 4 μM Ev1 protein and a 4 μM Rad51B protein. Further, lanes 1-4 indicate the results of experiments that were carried out in the presence of ATP, and lanes 5-8 indicate the results of experiments that were carried out in the absence of ATP. FIG. 8b shows the results obtained by examining the influence of a Rad51 protein on the activity of an Ev1 protein. Lanes 1 and 5 indicate the results of control experiments in which neither the Ev1 nor the Rad51 protein was used. Lanes 2 and 6 indicate the results of experiments that were carried out using a 4 μM Rad51 protein in the absence of the Ev1 protein. Lanes 3 and 7 indicate the results of experiments that were carried out using a 4 μM Ev1 protein in the absence of the Rad51 protein. Lanes 4 and 8 indicate the results of experiments that were carried out using a 4 μM Ev1 protein and a 4 μM Rad51 protein. Further, lanes 1-4 indicate the results of experiments that were carried out in the presence of ATP, and lanes 5-8 indicate the results of experiments that were carried out in the absence of ATP.

FIG. 9 shows the results obtained by examining the concentration-dependent effect of a Rad51B protein to promote the catenation activity of an Ev1 protein. The Rad51B protein was mixed in a concentration of 0.5 to 8 μM with a 4 μM Ev1 protein, and the activity was then measured. Lane 1 indicates the results of a control experiment in which neither the Rad51B protein nor the Ev1 protein was added, and lane 8 indicates the results of a control experiment in which a 8 μM Rad51B protein was added and no Ev1 protein was added. Lanes 2-7 indicate the results of experiments in which the Rad51B protein was added in concentrations of 0 μM, 0.5 μM, 1.0 μM, 2.0 μM, 4.0 μM, and 8.0 μM, respectively, in the presence of 4 μM Ev1.

FIG. 10 shows the results of purification of an Ev1 (1-221) mutant. FIG. 10a shows a process of purifying the Ev1 (1-221) mutant. FIG. 10b shows the results obtained by subjecting the sample in each purification step to 15% SDS-PAGE and then staining the resultant sample with Coomassie Brilliant Blue. Lane 1 indicates a molecular weight marker. Lanes 2 and 3 indicate a whole host cell extract before and after addition of IPTG, respectively. Lanes 4-7 indicate an Ni-NTA agarose fraction, a hydroxyapatite pass-through fraction, a fraction after a thrombin treatment, and a Superdex200 peak fraction, respectively.

FIG. 11 shows the results of purification of an Ev1 (222-418) mutant. FIG. 11a shows a process of purifying the Ev1 (222-418) mutant. FIG. 11b shows the results obtained by subjecting the sample in each purification step to 15% SDS-PAGE and then staining the resultant sample with Coomassie Brilliant Blue. Lane 1 indicates a molecular weight marker. Lanes 2 and 3 indicate a whole host cell extract before and after addition of IPTG, respectively. Lanes 4-7 indicate an Ni-NTA agarose fraction, a hydroxyapatite peak fraction, a fraction after a thrombin treatment, and a Superdex200 peak fraction, respectively.

FIG. 12 shows the results obtained by examining the activity of the EVH2 domain of an Ev1 protein. FIG. 12a is a schematic view showing the domain structure of the Ev1 protein and the deletion mutants used in the present example. EVH1, a proline-rich region, and EVH2 are shown in the figure. FIG. 12b shows the results obtained by analyzing the activities of Ev1 deletion mutants. Lanes 1-4 indicate the results obtained by examining such activity in the absence of protein, in the presence of the Ev1 protein (4 μM), in the presence of the Ev1 (1-221) mutant (4 μM), and in the presence of the Ev1 (222-418) mutant (4 μM), respectively. FIG. 12c shows the results obtained by analyzing the influence of a Rad51B protein on the activities of Ev1 deletion mutants. Lane 1 indicates the results of an experiment in which no protein was used. Lane 2 indicates the results of an experiment in which only the Rad51B protein was added. Lanes 3 and 4, lanes 5 and 6, and lanes 7 and 8 indicate the results of experiments in which the Ev1 protein (4 μM), the Ev1 (1-221) mutant (4 μM), and the Ev1 (222-418) mutant (4 μM) were added, respectively. Moreover, lanes 4, 6, and 8 indicate the results of experiments in which the Rad51B protein (4 μM) was further added.

FIG. 13 shows the results regarding the effect of the coexistence of a DNA topoisomerase type I protein and an Ev1 protein to promote the catenation of a single-stranded DNA. FIG. 13a shows the effect of TopoI (derived from Escherichia coli) to promote the catenation of a single-stranded DNA. Lane 1 indicates the results of a negative control in which only a single-stranded DNA was reacted. Lane 2 indicates the results obtained by reacting a 1 μM Ev1 protein with a single-stranded DNA. Lane 3 indicates the results obtained by reacting 5 U TopoI (E. coli, New England Biolabs) with a single-stranded DNA. Lane 4 indicates the results obtained by reacting a single-stranded DNA in the coexistence of a 1 μM Ev1 protein and 0.05 U TopoI. Lane 5 indicates the results obtained by reacting a single-stranded DNA in the coexistence of a 1 μM Ev1 protein and 0.5 U TopoI. Lane 6 indicates the results obtained by reacting a single-stranded DNA in the coexistence of a 1 μM Ev1 protein and 5 U TopoI. FIG. 13b shows the effect of hsTopoI (derived from a human) to promote the catenation of a single-stranded DNA. Lane 1 indicates the results of a negative control in which only a single-stranded DNA was reacted. Lane 2 indicates the results obtained by reacting a 0.5 μM Ev1 protein with a single-stranded DNA. Lane 3 indicates the results obtained by reacting 2.8 nM hsTopoI (human, Jena Bioscience) with a single-stranded DNA. Lane 4 indicates the results obtained by reacting a single-stranded DNA in the coexistence of a 0.5 μM Ev1 protein and 2.8 nM hsTopoI.

FIG. 14 shows the influence of aclarubicin on the activity of an Ev1 protein to catenate a single-stranded DNA. FIG. 14a shows the effect of aclarubicin to inhibit the single-stranded DNA catenation activity of the Ev1 protein. Lane 1 indicates the results of a negative control in which only a single-stranded DNA was reacted. Lane 2 indicates the results obtained by reacting a 4 μM Ev1 protein with a single-stranded DNA. Lane 3 indicates the results obtained by reacting a 4 μM Ev1 protein and 1 μM aclarubicin with a single-stranded DNA. Lane 4 indicates the results obtained by reacting a 4 μM Ev1 protein and 5 μM aclarubicin with a single-stranded DNA. Lane 5 indicates the results obtained by reacting a 4 μM Ev1 protein and 10 μM aclarubicin with a single-stranded DNA. Lane 6 indicates the results obtained by reacting a 4 μM Ev1 protein and 20 μM aclarubicin with a single-stranded DNA. Lane 7 indicates the results obtained by reacting 20 μM aclarubicin with a single-stranded DNA. FIG. 14b shows the influence of aclarubicin on the DNA binding activity of an Ev1 protein. The experiments were carried out in the same manner as that of FIG. 14a with the exception that the concentration of the reacted Ev1 protein was set at 0.3 μM.

FIG. 15 shows the influence of dequalinium on the activity of an Ev1 protein to catenate a single-stranded DNA. FIG. 15a shows the effect of dequalinium to inhibit the single-stranded DNA catenation activity of the Ev1 protein. Lane 1 indicates the results of a negative control in which only a single-stranded DNA was reacted. Lane 2 indicates the results obtained by reacting a 4 μM Ev1 protein with a single-stranded DNA. Lane 3 indicates the results obtained by reacting a 4 μM Ev1 protein and 1 μM dequalinium with a single-stranded DNA. Lane 4 indicates the results obtained by reacting a 4 μM Ev1 protein and 5 μM dequalinium with a single-stranded DNA. Lane 5 indicates the results obtained by reacting a 4 μM Ev1 protein and 10 μM dequalinium with a single-stranded DNA. Lane 6 indicates the results obtained by reacting a 4 μM Ev1 protein and 20 μM dequalinium with a single-stranded DNA. Lane 7 indicates the results obtained by reacting 20 μM dequalinium with a single-stranded DNA. FIG. 15b shows the influence of dequalinium on the DNA binding activity of an Ev1 protein. The experiments were carried out in the same manner as that of FIG. 15a with the exception that the concentration of the reacted Ev1 protein was set at 0.3 μM.

FIG. 16 shows the influence of DIDS on the activity of an Ev1 protein to catenate a single-stranded DNA. FIG. 16a shows the effect of DIDS to inhibit the single-stranded DNA catenation activity of the Ev1 protein. Lane 1 indicates the results of a negative control in which only a single-stranded DNA was reacted. Lane 2 indicates the results obtained by reacting a 4 μM Ev1 protein with a single-stranded DNA. Lane 3 indicates the results obtained by reacting a 4 μM Ev1 protein and 1 μM DIDS with a single-stranded DNA. Lane 4 indicates the results obtained by reacting a 4 μM Ev1 protein and 5 μM DIDS with a single-stranded DNA. Lane 5 indicates the results obtained by reacting a 4 μM Ev1 protein and 10 μM DIDS with a single-stranded DNA. Lane 6 indicates the results obtained by reacting a 4 μM Ev1 protein and 20 μM DIDS with a single-stranded DNA. Lane 7 indicates the results obtained by reacting 20 μM DIDS with a single-stranded DNA. FIG. 16b shows the influence of DIDS on the DNA binding activity of an Ev1 protein. The experiments were carried out in the same manner as that of FIG. 16a with the exception that the concentration of the reacted Ev1 protein was set at 0.3 μM.

FIG. 17 shows the influence of β-rubromycin on the activity of an Ev1 protein to catenate a single-stranded DNA. FIG. 17a shows the effect of β-rubromycin to inhibit the single-stranded DNA catenation activity of the Ev1 protein. Lane 1 indicates the results of a negative control in which only a single-stranded DNA was reacted. Lane 2 indicates the results obtained by reacting a 4 μM Ev1 protein with a single-stranded DNA. Lane 3 indicates the results obtained by reacting a 4 μM Ev1 protein and 1 μM β-rubromycin with a single-stranded DNA. Lane 4 indicates the results obtained by reacting a 4 μM Ev1 protein and 5 μM β-rubromycin with a single-stranded DNA. Lane 5 indicates the results obtained by reacting a 4 μM Ev1 protein and 10 μM β-rubromycin with a single-stranded DNA. Lane 6 indicates the results obtained by reacting a 4 μM Ev1 protein and 20 μM β-rubromycin with a single-stranded DNA. Lane 7 indicates the results obtained by reacting 20 μM β-rubromycin with a single-stranded DNA. FIG. 17b shows the influence of β-rubromycin on the DNA binding activity of an Ev1 protein. The experiments were carried out in the same manner as that of FIG. 17a with the exception that the concentration of the reacted Ev1 protein was set at 0.3 μM.

FIG. 18 shows the influence of 3-ATA on the activity of an Ev1 protein to catenate a single-stranded DNA. FIG. 18a shows the effect of 3-ATA to inhibit the single-stranded DNA catenation activity of the Ev1 protein. Lane 1 indicates the results of a negative control in which only a single-stranded DNA was reacted. Lane 2 indicates the results obtained by reacting a 4 μM Ev1 protein with a single-stranded DNA. Lane 3 indicates the results obtained by reacting a 4 μM Ev1 protein and 1 μM 3-ATA with a single-stranded DNA. Lane 4 indicates the results obtained by reacting a 4 μM Ev1 protein and 5 μM 3-ATA with a single-stranded DNA. Lane 5 indicates the results obtained by reacting a 4 μM Ev1 protein and 10 μM 3-ATA with a single-stranded DNA. Lane 6 indicates the results obtained by reacting a 4 μM Ev1 protein and 20 μM 3-ATA with a single-stranded DNA. Lane 7 indicates the results obtained by reacting 20 μM 3-ATA with a single-stranded DNA. FIG. 18b shows the influence of 3-ATA on the DNA binding activity of an Ev1 protein. The experiments were carried out in the same manner as that of FIG. 18a with the exception that the concentration of the reacted Ev1 protein was set at 0.3 μM.

### BEST MODE FOR CARRYING OUT THE INVENTION

A first embodiment of the present invention relates to “a composition for cleaving a single-stranded DNA and/or binding the 5′-terminus of such single-stranded DNA to the 3′-terminus thereof, which comprises an Ev1 protein described in the following (a) or (b):

(a) a protein having the amino acid sequence shown in SEQ ID NO: 2, 4, 6, 8, 10, 12, or 20; or
(b) a polypeptide, which has an amino acid sequence comprising a substitution, deletion, or insertion of one or several amino acids with respect to the amino acid sequence shown in SEQ ID NO: 2, 4, 6, 8, 10, 12, or 20, and which has activity of cleaving a single-stranded DNA and binding the 5′-terminus of such single-stranded DNA to the 3′-terminus thereof.”

The term “Ev1 protein” is used herein to mean a protein having an amino acid sequence identical to or substantially identical to the amino acid sequence shown in SEQ ID NO: 2, 4, 6, 8, 10, or 12. The description “a protein having an amino acid sequence . . . substantially identical to . . . ” is used herein to mean a protein, which has an amino acid sequence showing amino acid identity of approximately 60% or more, preferably approximately 70% or more, more preferably approximately 80%, 81%, 82%, 83%, 84%, 85%, 86%, 87%, 88%, 89%, 90%, 91%, 92%, 93%, 94%, 95%, 96%, 97% or 98%, and most preferably approximately 99%, with the amino acid sequence shown in SEQ ID NO: 2, 4, 6, 8, 10, or 12, and which has activity of cleaving a single-stranded DNA and binding the 5′-terminus of such single-stranded DNA to the 3′-terminus thereof.

It is to be noted that the terms “polypeptide” and “protein” have the same meanings in the present specification, unless otherwise specified.

Alternatively, the protein having an amino acid sequence substantially identical to the amino acid sequence shown in SEQ ID NO: 2, 4, 6, 8, 10, or 12 may be a protein, which has an amino acid sequence comprising a deletion, substitution, or addition of one or several amino acids (preferably approximately 1 to 30, more preferably approximately 1 to 10, and further preferably 1 to 5 amino acids) with respect to the amino acid sequence shown in SEQ ID NO: 2, 4, 6, 8, 10, or 12, and which has activity of cleaving a single-stranded DNA and binding the 5′-terminus of such single-stranded DNA to the 3′-terminus thereof.

The aforementioned deletion, addition and substitution of amino acids may be present in an isolated, native polypeptide. Otherwise, a gene encoding the protein of the present invention may be modified by a method known in the present technical field, so that such deletion, addition or substitution of amino acids may be newly introduced into a protein. For example, substitution of specific amino acid residue(s) may be carried out by substituting nucleotides with other nucleotides according to a known method such as a Gupped duplex method or a Kunkel method, or a method equivalent thereto, using a commercially available kit (for example, MutanTM-G (TAKARA), MutanTM-K (TAKARA), etc.).

The C-terminus of the protein used in the present invention is generally a carboxyl group (—COOH) or carboxylate (—COO—). However, such carboxyl group may be chemically modified with an amide (—CONH₂), an ester (—COOR), or the like. Herein, R in such ester includes C₁₋₆ alkyl groups (for example, methyl, ethyl, n-propyl, isopropyl, and n-butyl), C₃₋₈ cycloalkyl groups (for example, cyclopentyl and cyclohexyl), C₁₋₆ aryl groups (for example, phenyl and α-naphthyl), phenyl-C₁₋₂ alkyl groups (for example, benzyl and phenethyl), α-naphthyl-C₁₋₂ alkyl groups (for example, α-naphthylmethyl), and other groups. Otherwise, there may also be used a pivaloyloxymethyl ester, which has been generally known as an ester for oral use. When the Ev1 protein of the present invention has a carboxyl group not only at the C-terminus thereof but also in the polypeptide chain thereof, an amidated or esterified carboxyl group is also included in the protein of the present invention. Examples of such ester include the above-described esters. Likewise, the N-terminus of the protein of the present invention is generally an amino group (—NH₂). However, such amino group may be chemically modified with a C₁₋₆ acyl group such as a formyl group or an acetyl group. Moreover, the protein of the present invention also includes: a protein in which a glutamyl group generated as a result of the cleavage of the N-terminal side in vivo is converted to pyroglutamic acid; a protein in which a substituent on the side chain of an intramolecular amino acid (for example, —OH, —SH, an amino group, an imidazole group, an indole group, a guanidino group, etc.) is chemically modified with a suitable functional group (for example, formyl, acetyl, etc.); and a sugar chain-binding protein.

A peptide having a partial amino acid sequence contained in the Ev1 protein of the present invention (which is also referred to as a “partial peptide”) may also be included in the composition of the present invention, as long as the partial peptide has activity of cleaving a single-stranded DNA and binding the 5′-terminus of such single-stranded DNA to the 3′-terminus thereof. An example of such partial peptide is a polypeptide having an amino acid sequence identical to or substantially identical to the amino acid sequence shown in SEQ ID NO: 20. The description “a polypeptide having an amino acid sequence . . . substantially identical to . . . ” is used herein to mean a polypeptide, which has an amino acid sequence showing amino acid identity of approximately 60% or more, preferably approximately 70% or more, more preferably approximately 80%, 81%, 82%, 83%, 84%, 85%, 86%, 87%, 88%, 89%, 90%, 91%, 92%, 93%, 94%, 95%, 96%, 97% or 98%, and most preferably approximately 99%, with the amino acid sequence shown in SEQ ID NO: 20, and which has activity of cleaving a single-stranded DNA and binding the 5′-terminus of such single-stranded DNA to the 3′-terminus thereof

Alternatively, the polypeptide having an amino acid sequence substantially identical to the amino acid sequence shown in SEQ ID NO: 20 may be a polypeptide, which has an amino acid sequence comprising a deletion, substitution, or addition of one or several amino acids (preferably approximately 1 to 30, more preferably approximately 1 to 10, and further preferably 1 to 5 amino acids) with respect to the amino acid sequence shown in SEQ ID NO: 20, and which has activity of cleaving a single-stranded DNA and binding the 5′-terminus of such single-stranded DNA to the 3′-terminus thereof.

The aforementioned deletion, addition and substitution of amino acids may be present in an isolated, native polypeptide. Otherwise, a gene encoding the protein of the present invention may be modified by a method known in the present technical field, so that such deletion, addition or substitution of amino acids may be newly introduced into a protein. For example, substitution of specific amino acid residue(s) may be carried out by substituting nucleotides with other nucleotides according to a known method such as a Gupped duplex method or a Kunkel method, or a method equivalent thereto, using a commercially available kit (for example, MutanTM-G (TAKARA), MutanTM-K (TAKARA), etc.).

The Ev1 protein or a partial peptide thereof may be obtained from a natural source, or it may be obtained in the form of a recombinant. In order to obtain such recombinant, a recombinant vector is necessary for expression of the Ev1 protein or a partial peptide thereof. Moreover, such recombinant vector may be contained in the composition of the present invention. A nucleic acid encoding the Ev1 protein or a partial peptide thereof is inserted into the recombinant vector, such that the nucleic acid can be expressed therein. Herein, the description “can be expressed” is used to mean a state in which a nucleic acid encoding an Ev1 protein having a desired amino acid sequence or a partial peptide thereof, with a proper reading frame, is inserted into an expression vector, such that the Ev1 protein or a partial peptide thereof can be expressed therein, and in which other necessary components such as a promoter and a selective marker are also constructed in the vector, such that they can properly function.

The term “a nucleic acid encoding the Ev1 protein” is used herein to include, not only a nucleic acid having the nucleotide sequence shown in SEQ ID NO: 1, 3, 5, 7, 9, or 11, but also a nucleic acid, which hybridizes under stringent conditions with a nucleic acid having a nucleotide sequence complementary to the nucleotide sequence shown in SEQ ID NO: 1, 3, 5, 7, 9, or 11, and which encodes a polypeptide having activity of cleaving a single-stranded DNA and binding the 5′-terminus of such single-stranded DNA to the 3′-terminus thereof.

An example of DNA capable of hybridizing under stringent conditions with the nucleic acid having the nucleotide sequence shown in SEQ ID NO: 1, 3, 5, 7, 9, or 11 is a nucleic acid having a nucleotide sequence showing polynucleotide sequence homology of preferably approximately 70% or more, more preferably approximately 80%, 81%, 82%, 83%, 84%, 85%, 86%, 87%, 88%, 89%, 90%, 91%, 92%, 93%, 94%, 95%, 96%, 97% or 98%, and most preferably approximately 99%, with the nucleotide sequence shown in SEQ ID NO: 1, 3, 5, 7, 9, or 11.

The description “a nucleic acid encoding a partial peptide of the Ev1 protein” is used herein to include, not only a nucleic acid having the nucleotide sequence shown in SEQ ID NO: 19, but also a nucleic acid, which hybridizes under stringent conditions with a nucleic acid having a nucleotide sequence complementary to the nucleotide sequence shown in SEQ ID NO: 19 and which encodes a polypeptide having activity of cleaving a single-stranded DNA and binding the 5′-terminus of such single-stranded DNA to the 3′-terminus thereof.

An example of DNA capable of hybridizing under stringent conditions with the nucleic acid having the nucleotide sequence shown in SEQ ID NO: 19 is a nucleic acid having a nucleotide sequence showing polynucleotide sequence homology of preferably approximately 70% or more, more preferably approximately 80%, 81%, 82%, 83%, 84%, 85%, 86%, 87%, 88%, 89%, 90%, 91%, 92%, 93%, 94%, 95%, 96%, 97% or 98%, and most preferably approximately 99%, with the nucleotide sequence shown in SEQ ID NO: 19.

The term “stringent conditions” is used herein to mean conditions for hybridization, which are easily determined by persons skilled in the art. Such stringent conditions are various empirical conditions that are generally dependent on the length of a probe, a washing temperature, and a salt concentration. In general, as a probe becomes longer, a temperature necessary for appropriate annealing also increases. On the other hand, as a probe becomes shorter, such temperature decreases. Hybrid formation generally depends on the ability of a nucleic acid in which a complementary strand is allowed to reanneal under temperature conditions slightly lower than the melting point thereof.

Specific examples of low stringent conditions include washing a filter at a temperature between 37° C. and 42° C. in a 0.1×SSC and 0.1% SDS solution in the step of washing the filter after completion of the hybridization. Specific examples of high stringent conditions include washing a filter at 65° C. in a 5×SSC and 0.1% SDS solution in such washing step. By further increasing such stringent conditions, a highly homologous polynucleotide can be obtained.

A nucleic acid encoding an Ev1 protein or a partial peptide thereof can be obtained from the cells of eukaryotes including mammals such as a human, a rat, a mouse, and a sheep, according to a common method. Alternatively, it is also possible to obtain the nucleic acid sequence information of the Ev1 protein, which has already been known, from database and the like, and to obtain an Ev1 gene derived from a desired organism species based on the sequence information. Such Ev1 gene can be cloned based on common knowledge in the present technical field. The gene can be obtained by preparing a cDNA library from cells that express the gene and then applying a common screening method. Alternatively, the gene may also be obtained by preparing RNA from cells that express the gene, synthesizing cDNA from the RNA using reverse transcriptase, then preparing PCR primers based on the gene sequence, and then amplifying the cDNA using the prepared PCR primers.

A recombinant vector, into which a nucleic acid encoding an Ev1 protein or a partial peptide thereof has been incorporated, can be obtained by ligating the nucleic acid to a suitable vector. When a recombinant vector is subjected to a cloning procedure, the type of the recombinant vector is not particularly limited, as long as it is able to replicate in a host. Moreover, as a vector used for expression of the Ev1 protein or a partial peptide thereof, a vector that is able to replicate in a host and allows a DNA fragment encoding the protein to express therein, such as promoter, can be used.

Examples of an available vector include plasmid DNA and phage DNA. Examples of such plasmid DNA include Escherichia coli-derived plasmids (for example, pBR322, pBR325, pUC118, pUC119, pUC18, pUC19, pCBD-C, pET15b, etc.), Bacillus subtilis-derived plasmids (for example, pUB110, pTP5, pC194, etc.), and yeast-derived plasmids (for example, YEp13, YEp24, YCp50, YIp30, etc.). An example of phage DNA is λ, phage. Moreover, animal virus vectors such as retrovirus or vaccinia virus, or insect virus vectors such as baculovirus or Togavirus, may also be used.

The type of a promoter used in the present invention is not particularly limited, as long as it is a promoter compatible with a host used in expression of a gene.

When an animal cell is used as a host, for example, available promoters include an SRα promoter, a CMV promoter, an SV40 promoter, an LTR promoter, an HSV-TK promoter, an EF-1α promoter, and the like.

When Escherichia coli is used as a host, available promoters include a tac promoter, a trp promoter, a lac promoter, a recA promoter, a λPL promoter, a lpp promoter, a T7 promoter, and the like. When Bacillus subtilis is used as a host, available promoters include an SPO1 promoter, an SPO2 promoter, a penP promoter, and the like.

When yeast is used as a host, available promoters include a PHO5 promoter, a PGK promoter, a GAP promoter, and an ADH promoter, and the like.

When an insect cell is used as a host, a polyhedrin promoter, a P10 promoter, and the like are preferable.

To the recombinant vector of the present invention, not only a nucleic acid sequence encoding an Ev1 protein or a portion thereof and a promoter sequence, but also a selective marker, a terminator, an enhancer, a splicing signal, a poly(A) addition signal, a ribosome binding sequence (SD sequence), an SV40 replication origin (SV40ori), and the like can be ligated.

The type of a selective marker is not limited. A hygromycin resistance marker (Hyg^{r}), a dihydrofolate reductase gene (dhfr), an ampicillin resistance gene (Amp^{r}), a kanamycin resistance gene (Kan^{r}), a neomycin resistance gene (Neo^{r}, G418), and the like can be used.

Moreover, for the purpose of facilitating isolation and purification of a recombinant protein, a tag sequence used for purification, such as a His tag, an HA tag, or GST, can be fused on the N-terminal side, C-terminal side, etc. of a protein to be expressed or a portion thereof.

When the host is Escherichia coli, an alkaline phosphatase signal, an OmpA signal, and the like can be used. When the host is Bacillus subtilis, an α-amylase signal sequence, a subtilis signal sequence, and the like can be used. When the host is yeast, an α-factor signal sequence, an invertase signal sequence, and the like can be used. When the host is an animal cell, an insulin signal sequence, an α-interferon signal sequence, and the like can be used, for example.

DNA encoding an Ev1 protein or a portion thereof can be inserted into the aforementioned vector by adding the cloned DNA to a linker, directly or after digesting it with restriction enzymes as desired, and then incorporating the resultant DNA into the restriction site or multicloning site of vector DNA. The thus ligated DNA may have ATG acting as a translation start codon on the 5′-terminal side thereof and may also have TAA, TGA, or TAG acting as a translation stop codon on the 3′-terminal side thereof. Such translation start codon and translation stop codon may also be added to the DNA, using an appropriate synthetic DNA adapter. It is necessary that DNA to be ligated be incorporated into the vector, so that the polypeptide of the present invention encoded in the DNA can be expressed in a host cell.

In order to allow an Ev1 protein or a partial peptide thereof to express, it is necessary that a suitable host cell be transformed with an expression vector, into which a nucleic acid encoding such protein or partial peptide has been inserted. The type of a host used in such transformation is not particularly limited, as long as it allows the Ev1 protein to express therein. Examples of such a host include: bacteria belonging to genus Escherichia such as Escherichia coli, genus Bacillus such as Bacillus subtilis, genus Pseudomonas such as Pseudomonas putida, or genus Rhizobium such as Rhizobium meliloti; yeasts such as Saccharomyces cerevisiae, Shizosaccharomyces pombe, or Pichia pastoris; monkey cells such as COS-7 and Vero; Chinese hamster ovary cells (CHO cells); and insect cells such as Sf9 or Sf21.

As a method of introducing a recombinant vector into Escherichia coli, a method using calcium ion, an electroporation method, and the like can be used. As a method of introducing a recombinant vector into yeast, an electroporation method, a spheroplast method, a lithium acetate method, and the like can be used. As a method of introducing a recombinant vector into an animal cell or an animal cell, a DEAE-dextran method, an electroporation method, a calcium phosphate method, a method using cationic lipids, and the like can be used.

An Ev1 protein or a partial peptide thereof can be produced by culturing a transformant, allowing the protein or the partial peptide thereof to express in the culture of the transformant, and then isolating the protein or the partial peptide thereof from the culture. The term “culture” is used herein to mean all of a culture supernatant, a cultured cell, a cultured cell mass, and a disintegrated product of such cell or cell mass.

As a medium for culturing a transformant using a microorganism such as Escherichia coli or yeast as a host, either a natural medium or a synthetic medium may be used, as long as it contains a carbon source, a nitrogen source, inorganic salts, and the like that can be assimilated by microorganisms, and it is able to efficiently culture the transformant. Examples of a carbon source used herein include carbohydrates such as glucose, fructose, sucrose, or starch, organic acids such as acetic acid or propionic acid, and alcohols such as ethanol or propanol. Examples of a nitrogen source used herein include the ammonium salts of inorganic acids or organic acids, such as ammonia, ammonium chloride, ammonium sulfate, ammonium acetate, or ammonium phosphate, other nitrogen-containing compounds, peptone, meat extract, and corn steep liquor. Examples of inorganic salts used herein include monopotassium phosphate, dipotassium phosphate, magnesium phosphate, magnesium sulfate, sodium chloride, ferrous sulfate, manganese sulfate, copper sulfate, and calcium carbonate.

The culture is carried out under conditions that are suitable for host cells. For example, as a medium used for culturing Escherichia coli, an LB medium, an M9 medium, and the like are preferable. In order to allow a promoter to act efficiently, an agent such as isopropyl-1-thio-β-D-galactoside or 3β-indolylacrylic acid may be added, as desired. In the case of Escherichia coli, the culture is generally carried out at approximately 15° C. to 37° C. for approximately 3 to 24 hours, and thereafter, aeration or stirring may also be carried out, if necessary. In a case in which the host is Bacillus subtilis, the culture is generally carried out at approximately 30° C. to 40° C. for approximately 6 to 24 hours, and thereafter, aeration or stirring may also be carried out, if necessary.

As a medium for culturing yeast, an SD medium or a YPD medium may be used. The pH of the medium is preferably adjusted to pH 5 to 8. The culture is generally carried out at approximately 20° C. to 35° C. for approximately 24 to 72 hours, and thereafter, aeration or stirring may also be carried out, if necessary. When a transformant whose host is an insect cell or an insect is cultured, a Grace's insect cell culture medium that contains bovine serum and the like may be used. The pH of the medium is preferably adjusted to pH 6.2 to 6.4. The culture is generally carried out at approximately 27° C. for approximately 3 to 5 days, and thereafter, aeration or stirring may also be carried out, if necessary.

When a transformant whose host is an animal cell is cultured, an MEM medium, a DMEM medium, an RPMI-1640 medium, or the like, which contains approximately 5% to 20% fetal bovine serum, is used. The pH is preferably pH 6 to 8. The culture is generally carried out at approximately 30° C. to 40° C. for approximately 15 to 60 hours, and thereafter, aeration or stirring may also be carried out, if necessary. An Ev1 protein or a partial peptide thereof may be separated and purified from the aforementioned culture by the following method, for example.

In order to extract such Ev1 protein or a partial peptide thereof from the cultured cell mass or the cultured cells, there may be applied, as appropriate, a method comprising collecting a cell mass or cells according to a known method after completion of the culture, suspending such cell mass or cells in a suitable buffer solution, and then disintegrating such cell mass or cells with the use of ultrasonic wave, lysozyme, and/or a freezing and thawing method, followed by centrifugation or filtration, so as to obtain a crude extract of the Ev1 protein or the partial peptide thereof. The buffer solution may comprise protein denaturants such as urea or guanidine hydrochloride, or surfactants such as Triton X-100. When the Ev1 protein or the partial peptide thereof is secreted into the culture solution, a culture supernatant is separated from a cell mass or cells according to a known method after completion of the culture, and such supernatant is then collected. The Ev1 protein or the partial peptide thereof contained in the thus obtained culture supernatant or extract may be purified by the appropriate combined use of known separation and purification methods. Known separation and purification methods that can be used herein include: methods utilizing solubility, such as a salting-out method and a solvent precipitation method; methods mainly utilizing a difference in molecular weight, such as a dialysis method, an ultrafiltration method, a gel filtration method, and SDS-PAGE; methods utilizing a difference in electric charge, such as ion exchange chromatography; methods utilizing specific affinity, such as affinity chromatography; methods utilizing a difference in hydrophobicity, such as reversed-phase high performance liquid chromatography; methods utilizing a difference in isoelectric point, such as an isoelectric focusing method; and other methods.

A composition comprising the Ev1 protein, the partial peptide thereof, or an expression vector containing such protein or peptide, can be used to cleave a single-stranded DNA and/or to bind the 5′-terminus of such single-stranded DNA to the 3′-terminus thereof. The present composition may comprise all substances necessary for the cleavage or binding of a single-stranded DNA, as well as the Ev1 protein, the partial peptide thereof, the expression vector therefor, and the like. For example, the present composition may comprise metal ion such as Mg²⁺, ATP, and auxiliary substances such as a buffer for keeping pH constant.

In another embodiment, the present invention provides a composition, which further comprises a Rad51B protein or an expression vector for the Rad51B protein, as well as the aforementioned active ingredients, auxiliary substances, and others.

The term “Rad51B protein” is used herein to mean a protein having an amino acid sequence identical to or substantially identical to the amino acid sequence shown in SEQ ID NO: 14, 16, or 18. The description “protein having an amino acid sequence . . . substantially identical to . . . ” is used herein to mean a protein, which has an amino acid sequence showing amino acid identity of approximately 60% or more, preferably approximately 70% or more, more preferably approximately 80%, 81%, 82%, 83%, 84%, 85%, 86%, 87%, 88%, 89%, 90%, 91%, 92%, 93%, 94%, 95%, 96%, 97% or 98%, and most preferably approximately 99%, with the amino acid sequence shown in SEQ ID NO: 14, 16, or 18, and which has ATPase activity and activity of binding to a Holiday structure that is a DNA structure specific to homologous recombination.

Alternatively, the protein having an amino acid sequence substantially identical to the amino acid sequence shown in SEQ ID NO: 14, 16, or 18 may be a protein, which has an amino acid sequence comprising a deletion, substitution, or addition of one or several amino acids (preferably approximately 1 to 30, more preferably approximately 1 to 10, and further preferably 1 to 5 amino acids) with respect to the amino acid sequence shown in SEQ ID NO: 14, 16, or 18, and which has ATPase activity and activity of binding to a Holiday structure that is a DNA structure specific to homologous recombination.

The aforementioned deletion, addition and substitution of amino acids may be present in an isolated, native polypeptide. Otherwise, a gene encoding the protein of the present invention may be modified by a method known in the present technical field, so that such deletion, addition or substitution of amino acids may be newly introduced into a protein. For example, substitution of specific amino acid residue(s) may be carried out by substituting nucleotides with other nucleotides according to a known method such as a Gupped duplex method or a Kunkel method, or a method equivalent thereto, using a commercially available kit (for example, MutanTM-G (TAKARA), MutanTM-K (TAKARA), etc.).

The Rad51B protein may be obtained from native environment, or may be obtained by allowing a recombinant protein to express. When a recombinant Rad51B protein is allowed to express, an expression vector and components necessary for such expression may be selected in accordance with the above-described method for expressing an Ev1 protein. Such recombinant protein may be obtained in accordance with the aforementioned method for obtaining a recombinant Ev1 protein.

In addition, in a further embodiment, the present invention provides a composition, which further comprises a DNA topoisomerase type I protein or an expression vector for the DNA topoisomerase type I protein, as well as the Ev1 protein or the expression vector for the Ev1 protein. In general, the term “DNA topoisomerase type I” is also referred to as “TopoI.” However, it is unnecessary to attach our mind to such common name. The DNA topoisomerase type I has activity of introducing a nick into one strand of a circular (double-stranded) DNA, passing the other stand through it, and then rebinding the nick. Thus, the DNA topoisomerase type I is considered to include all enzymes having activity of alleviating the supercoiling of DNA. Accordingly, the “DNA topoisomerase type I” of the present invention also includes those derived from either prokaryotes or eukaryotes. In addition, enzymes acting to relax either positive or negative supercoiling of DNA and enzymes acting to relax both positive and negative supercoiling of DNA are also included in the DNA topoisomerase type I of the present invention. A DNA topoisomerase type I protein may be obtained from native environment, or may be obtained by allowing a recombinant protein to express. Otherwise, commercially available products may be purchased. When a recombinant DNA topoisomerase type I protein is allowed to express, an expression vector and components necessary for such expression may be selected in accordance with the above-described method for expressing an Ev1 protein. Such recombinant protein may be obtained in accordance with the aforementioned method for obtaining a recombinant Ev1 protein.

In a further embodiment, the present invention provides a composition for inhibiting Ev1 activity. The Ev1 protein has activity of cleaving and/or binding a single-stranded DNA. When such Ev1 activity is suppressed or regulated, at the correct time, by a composition for inhibiting such activity, the formation of a single-stranded DNA having a more desired form can be achieved. Compounds contained as active ingredients in the composition for inhibiting Ev1 activity include aclarubicin, dequalinium, DIDS (disodium salt), β-rubromycin, and/or 3-ATA (3-amino-9-thio(10H)-acridone). These compounds may be contained in the composition for inhibiting Ev1 activity in the form of salts.

The composition of the present invention may further comprise additive substances that are generally necessary for preparation of reagents, such as a buffer used to adjust pH, salts used to adjust ionic strength, and a protease inhibitor, as well as active ingredients such as a compound, a protein, and DNA. Such additive substances may be appropriately selected and used by persons skilled in the art, depending on the intended use of the composition of the present invention.

In a further embodiment, the present invention provides a method for producing a single-stranded DNA molecular weight marker using the composition of the present invention, and a single-stranded DNA molecular weight marker produced by the aforementioned method. The single-stranded molecular weight marker of the present invention is produced by preparing single-stranded DNA molecules each having a different molecular weight that is equal to the integral multiple of the molecular weight of another single-stranded DNA having a known molecular weight. Accordingly, the molecular weight marker of the present invention is provided as a mixture of a single-stranded DNA used as a starting substance and/or single-stranded DNA molecules each having a different molecular weight that is equal to the integral multiple of the molecular weight of the aforementioned single-stranded DNA. Moreover, the molecular weight marker as such mixture is separated by agarose gel electrophoresis, and a single-stranded DNA corresponding to each molecular weight is cleaved, so that a single-stranded DNA having a specific molecular weight can be produced. Any type of single-stranded DNA can be used in the method for producing the single-stranded DNA molecular weight marker of the present invention. A circular single-stranded DNA is preferably used.

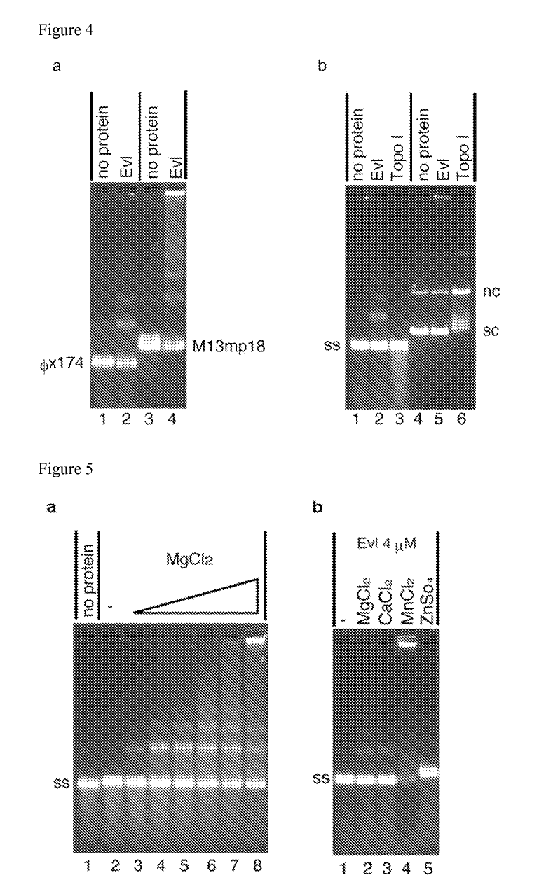

As described above, using a composition comprising the Ev1 protein of the present invention, a single-stranded DNA can be cleaved and can be then bound again. Thus, according to the present invention, single-stranded DNA molecules having various lengths (or molecular weights) can be produced. For example, referring to the example section of the present invention, a molecular weight marker consisting of 5368×n nucleotides (wherein n represents an integer of 1 or greater) can be produced by allowing a composition comprising the Ev1 protein of the present invention to act on a φ×174 circular single-stranded DNA (5368 nucleotides), as shown in FIGS. 14a, 15a, 16a, 17a, and 18a. (In FIGS. 14a, 15a, 16a, 17a, and 18a, single-stranded DNA is schematically shown with oval shapes on the right side of a photograph of gel. A single oval shape indicates the position of a single-stranded DNA consisting of 5368 nucleotides, two oval shapes indicate the position of a single-stranded DNA consisting of 5368×2 nucleotides, and three oval shapes indicate the position of a single-stranded DNA consisting of 5368×3 nucleotides.) Similarly, using a single-stranded DNA having a different molecular weight, such as M13 mp18 (7250 nucleotides), a molecular weight marker of 7250×n (wherein n represents an integer of 1 or greater) can be produced (please refer to FIG. 4).

Using the thus produced single-stranded DNA molecular weight marker, when a single-stranded DNA is extracted with helper phage or the like, the molecular weight of the extracted DNA can be examined

Even after a single-stranded DNA marker produced with the composition of the present invention has been precipitated with ethanol and has been then freeze-dried, its molecular weight does not change. Thus, the present single-stranded DNA marker can be used as a stable single-stranded DNA molecular weight marker.

Examples will be given below. However, these examples are not intended to limit the scope of the present invention.

### Examples

### 1. Purification of Human Ev1 Protein

An Ev1 protein (NCBI accession No. AAF21709) DNA fragment (for example, SEQ ID NO: 1) was isolated from a human cDNA pool (Clontech) by a PCR method, and it was then cloned into the NdeI site of a pET15b vector (Novagen). In this construct, a His tag was fused on the N-terminal side of the isolated gene. The Ev1 protein was allowed to express using an E. coli BL21 (DE3) codon plus-RP strain (Stratagene), and it was then purified via 4 steps including a step of removing 6×His tag. First, cells that expressed the Ev1 protein were suspended in buffer A (20 mM potassium phosphate (pH 8.5), 700 mM NaCl, 5 mM 2-mercaptoethanol, 10 mM imidazole, and 10% glycerol), and the cells were then disintegrated with an ultrasonic disintegrator. The obtained cell disintegrated solution was centrifuged at 30,000×g at 4° C. for 20 minutes, and the obtained supernatant was gently mixed with 8 ml of Ni-NTA agarose beads (QIAGEN). Thereafter, the obtained mixture was allowed to bind to the His tag-fused Ev1 protein (His-Ev1) by a batch method at 4° C. for 1 hour.

Ev1-bound beads were washed with 80 ml of buffer B (20 mM potassium phosphate (pH 8.5), 700 mM NaCl, 5 mM 2-mercaptoethanol, 30 mM imidazole, and 10% glycerol). The beads were then washed with 80 ml of buffer C (20 mM potassium phosphate (pH 8.5), 700 mM NaCl, 5 mM 2-mercaptoethanol, 60 mM imidazole, and 10% glycerol), and were then washed with 80 ml of the buffer B again. Thereafter, an Econo-Column (Bio-Rad) was filled with the obtained Ev1-bound beads. The thus filled Ev1-bound beads were washed with 300 ml of buffer D (20 mM potassium phosphate (pH 8.5), 100 mM NaCl, 5 mM 2-mercaptoethanol, 30 mM imidazole, and 10% glycerol), and His-Ev1 was then eluted by linear concentration gradient elution with imidazole of 30 to 300 mM.

Buffer F (10 mM potassium phosphate (pH 8.5), 100 mM NaCl, 5 mM 2-mercaptoethanol, and 10% glycerol) was added in an equal amount to a fraction containing His-Ev1, and further, the obtained mixture was gently mixed with 10 ml of hydroxyapatite (BIO-RAD) by a batch method at 4° C. for 1 hour. Thereafter, resin was washed with 80 ml of buffer G (20 mM potassium phosphate (pH 8.5), 100 mM NaCl, 5 mM 2-mercaptoethanol, and 10% glycerol), and an Econo-Column was then filled with the resultant resin. The thus filled resin was washed with 300 ml of buffer H (10 mM potassium phosphate (pH 8.5), 225 mM NaCl, 5 mM 2-mercaptoethanol, and 10% glycerol), and His-Ev1 was then eluted by linear concentration gradient elution with NaCl of 225 to 1000 mM and potassium phosphate (pH 8.5) of 10 to 300 mM.

5 units of Thrombin Protease (GE Healthcare Bio-Sciences) was added per mg of the obtained His-Ev1, and the obtained mixture was then dialyzed at 4° C. against 4 L of buffer J (20 mM potassium phosphate (pH 8.5), 130 mM NaCl, 5 mM 2-mercaptoethanol, and 10% glycerol).

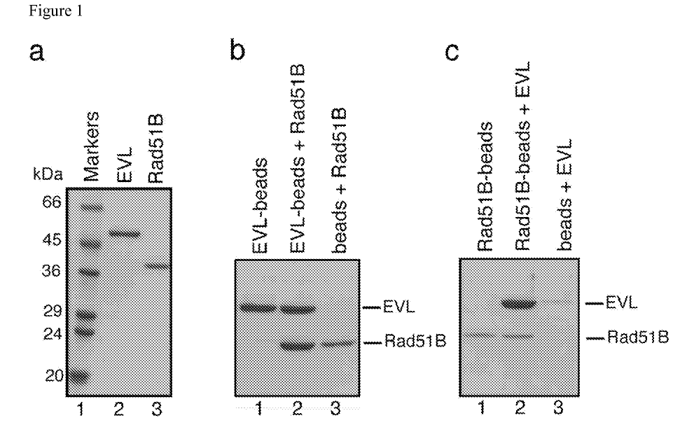

After removing the His tag, the Ev1 protein was further purified by Superdex200 gel filtration column (HiLoad 26/60 Superdex200 prep grade, GE Healthcare Bio-Sciences) chromatography. After completion of the purification, the Ev1 protein was dialyzed against buffer K (20 mM HEPES (pH 7.3), 100 mM NaCl, 5 mM 2-mercaptoethanol, and 30% glycerol) or against buffer L (20 mM potassium phosphate (pH 8.5), 700 mM NaCl, 5 mM 2-mercaptoethanol, and 30% glycerol). The resultant was preserved at −20° C. The concentration of the purified Ev1 protein was measured by a Bradford method using BSA as a standard. FIG. 1a shows the results of the 12% SDS-PAGE of the purified Ev1 protein.

### 2. Pull-Down Assay Using Ev1- or Rad51B-Bound Beads

A Rad51B protein was purified in accordance with the previously reported method (Yokoyama et al., J. Biol. Chem. 278: 2767-2772, 2003).

An Ev1 protein and a Rad51B protein were each allowed to bind to Affi-Gel 10 beads (Bio-Rad) in accordance with the instruction manual Ethanolamine (pH 8.0) was added thereto, so that the remaining ester residues had a final concentration of 100 mM. Thereafter, the obtained mixture was incubated at 4° C. overnight. The Affi-Gel 10-Ev1 beads were washed 3 times with 500 μl of washing buffer 1 (20 mM potassium phosphate (pH 8.5), 30% glycerol, 700 mM NaCl, 5 mM 2-mercaptoethanol, and 0.05% Triton X-100). The Affi-Gel 10-Rad51B beads were washed 3 times with washing buffer 2 (20 mM HEPES-NaOH (pH 7.3), 30% glycerol, 90 mM NaCl, 2 mM 2-mercaptoethanol, 0.1% Triton X-100, 2 mM ammonium sulfate, and 0.1 mM EDTA). Thereafter, a 50% suspension of such Affi-Gel 10-protein was prepared, and it was then preserved at 4° C.

In order to carry out a binding assay, an Affi-Gel 10-protein suspension (30 μl) was incubated with 10 μg of an Ev1 or Rad51B protein at room temperature for 150 minutes. Affi-Gel 10-Ev1 beads were washed 4 times with 100 μl of buffer 1 (20 mM potassium phosphate (pH 8.5), 30% glycerol, 700 mM NaCl, 5 mM 2-mercaptoethanol, and 0.3% Triton X-100). Affi-Gel 10-Rad51B beads were washed 4 times with 100 μl of buffer 2 (20 mM HEPES-NaOH (pH 7.3), 30% glycerol, 90 mM NaCl, 2 mM 2-mercaptoethanol, 2 mM ammonium sulfate, 0.1 mM EDTA, and 0.35% Triton X-100). An SDS-PAGE sample treatment buffer (2×) was directly mixed with the washed beads, and the obtained mixture was then subjected to a heat treatment at 100° C. for 2 minutes. The reaction product was separated by 12% SDS-PAGE, and the protein was then stained Coomassie Brilliant Blue.

As shown in FIG. 1b, the Rad51B protein was pulled downed by the Ev1-bound beads. In addition, the Ev1 protein was pulled down by the Rad51B-bound beads (FIG. 1c).

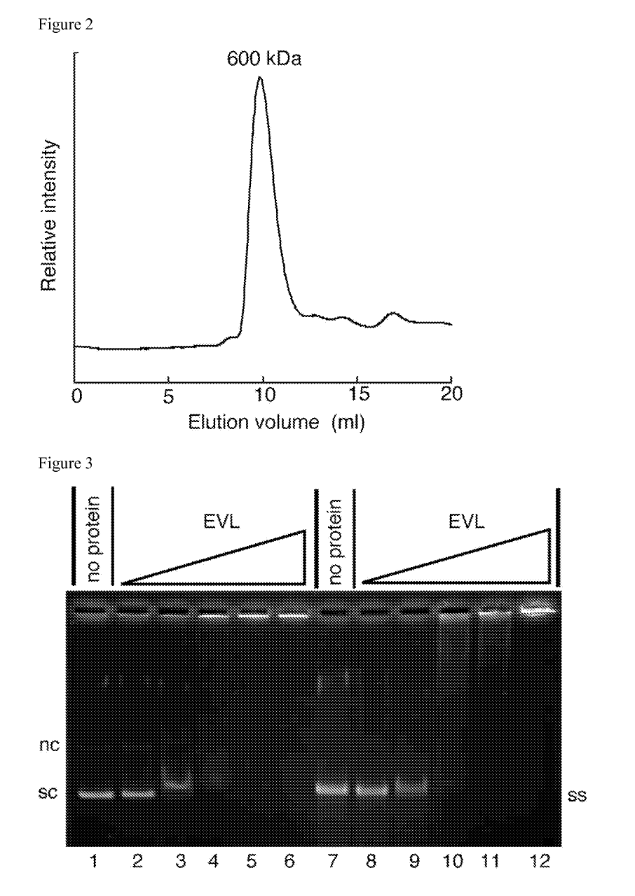

From these results, it became clear that the Ev1 protein directly binds to Rad51B. When such Ev1-bound beads were used in a pull-down assay, Rad51B bound to the Ev1-bound beads at a stoichiometric ratio of 1:1 (FIG. 1b). On the other hand, a large amount of Ev1 protein was precipitated together with the Rad51B-bound beads (FIG. 1c). Accordingly, it is considered that an Ev1 protein is polymerized with another Ev1 protein to form a complex. As a result of gel filtration analysis, the Ev1 protein was eluted at a fraction of approximately 600 kDa, and thus it was demonstrated that the Ev1 protein formed an approximately 13-mer multimer (FIG. 2).

### 3. DNA Binding Assay

Subsequently, the binding ability of the Ev1 protein to DNA was analyzed. The Ev1 protein was mixed with a φ×174 circular single-stranded DNA (40 μM) or a φ×174 supercoiled double-stranded DNA (10 μM) in 10 μl of a reaction solution (20 mM HEPES (pH 7.5), 1 mM DTT, 1 mM MgCl₂, and 100 mg/ml BSA), and the mixture was then reacted at 37° C. for 15 minutes. The reaction product was analyzed by 0.8% agarose gel electrophoresis (3 V/cm, 3 hours) using a 1×TAE (40 mM Tris-acetate and 1 mM EDTA) buffer. A band of DNA was stained with ethidium bromide (FIG. 3). As a result of the assay, it was found that the Ev1 protein efficiently binds to a single-stranded DNA and a supercoiled double-stranded DNA (FIG. 3).

### 4. Assay of Catenation of Single-Stranded DNA

A φ×174 or M13 mp18 circular single-stranded DNA (20 μM) was incubated at 37° C. in 20 mM HEPES-NaOH (pH 7.5), 1 mM DTT, 1 mM MgCl₂, and 0.1 mg/ml BSA. Thereafter, the sample was subjected to a deproteination treatment with 0.2% SDS and 1.3 mg/ml proteinase K. The obtained product was separated by 0.9% agarose gel electrophoresis, and a DNA band was then stained with SYBR Gold (Invitrogen).

After the Ev1 protein had been allowed to act on the aforementioned DNA molecules, the circular single-stranded DNA formed a multimer (FIG. 4a, lanes 2 and 4; and FIG. 4b, lane 2), but the supercoiled double-stranded DNA was not particularly changed (FIG. 4b, lane 5). The Ev1 protein did not induce a change in the topology of the supercoiled double-stranded DNA. Thus, it is considered that the activity of the Ev1 protein differs from the activity of topoisomerase I (FIG. 4b, lane 6).

Subsequently, the metal ion requirement of the Ev1 protein was examined. As a result, it was found that the Ev1 protein strongly requires Mg²⁺ (FIG. 5a). Even in the absence of Mg²⁺, the catenation activity of the Ev1 protein was observed. However, such activity was promoted by the presence of Mg²⁺. The optimal concentration of Mg²⁺ depends on reaction conditions (the concentrations of other ingredients, such as a glycerol concentration or a salt concentration). The concentration of Mg²⁺ is, for example, 0.1 mM to 20 mM, preferably 0.5 mM to 15 mM, more preferably 0.5 mM to 10 mM, and further preferably 0.5 mM to 2 mM. In addition, with regard to metal ions other than Mg²⁺, Ca²⁺ exhibited the same effect (FIG. 5b). Under the present experimental conditions, an enormous DNA complex-like product was formed in the case of adding Mn²⁺. In addition, Zn²⁺ did not have the same level of activity of promoting the activity of the Ev1 protein as that of Mg²⁺ (FIG. 5b).

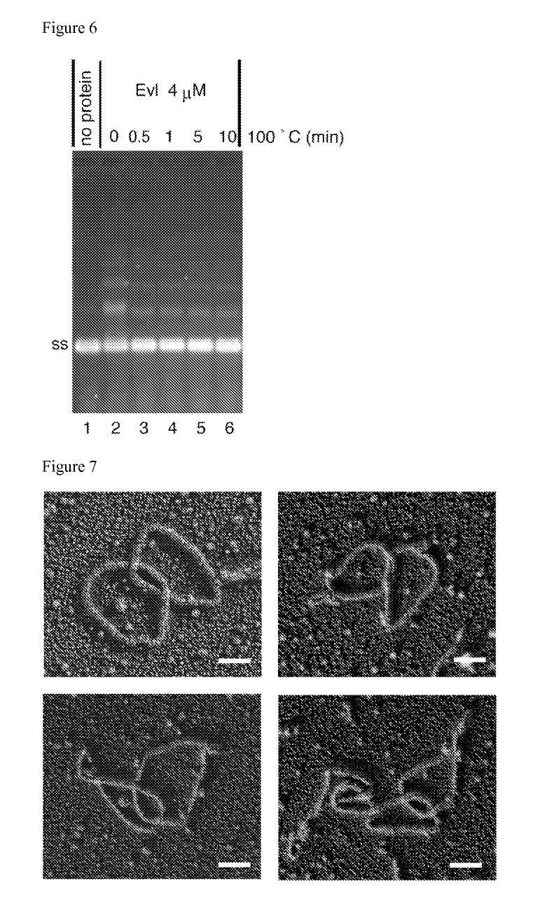

Moreover, the formed single-stranded DNA multimer was not eliminated, even after it had been treated at 100° C. for 10 minutes (FIG. 6). Thus, it is suggested that such multimer be a catemer of circular single-stranded DNA molecules catenated to one another to form a ring.

Subsequently, in order to visually understand a state in which circular single-stranded DNA molecules were catenated by the action of the Ev1 protein, a generated product was observed under an electron microscope.

### 5. Electron Microscope Observation

A single-stranded DNA catemer formed by the Ev1 protein was extracted by a phenol/chloroform method, and it was then recovered by ethanol precipitation. Subsequently, such single-stranded DNA catemer was coated with a RecA protein (New England Biolabs) in the absence of ATP, and it was then stained with 2% uranyl acetate on a carbon-coated copper grid. The stained sample was visualized by a rotary shadow method using tungsten, and it was then observed under a JEOL JEM2000 FX electron microscope.

As shown in FIG. 7, a circular single-stranded DNA catemer containing 2 or 3 single-stranded DNA molecules was observed.

As stated above, as a result of the assay of catenation of single-stranded DNA molecules and the observation under an electron microscope, it is considered that the Ev1 protein cleaves a circular single-stranded DNA and then binds the 5′-terminus thereof to the 3′-terminus thereof, so as to form a catemer. Accordingly, it can be concluded that the Ev1 protein has activity of cleaving a single-stranded DNA and binding the 5′-terminus of such single-stranded DNA to the 3′-terminus thereof.

It is to be noted that it has been revealed that such catenation occurs even in the case of linear single-stranded DNA molecules.

### 6. Influence of Rad51B

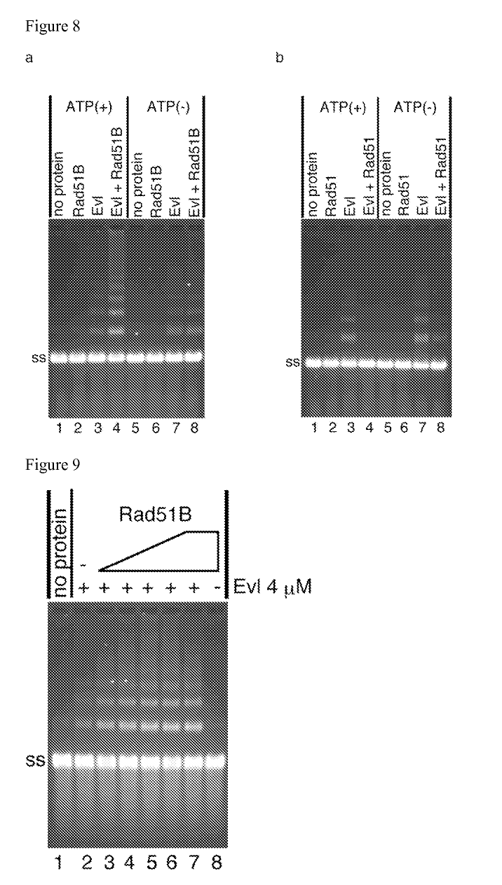

Subsequently, the influence of Rad51B on the catenation of single-stranded DNA molecules by the Ev1 protein was examined. As shown in FIG. 8a, it was found that the catenation of DNA molecules by the Ev1 protein was significantly promoted by Rad51B in the presence of ATP (lane 4). In contrast, such Rad51B-dependent promotion of catenation was not significant in the absence of ATP (FIG. 8a, lane 8). Accordingly, it is suggested that the ATP-bound Rad51B protein would promote the catenation of DNA molecules by the Ev1 protein. The effect of Rad51B to promote the activity of the Ev1 protein increases in an added Rad51B concentration dependent manner. However, when Rad51B was added in a concentration of 1 μM or higher to a 4 μM Ev1 protein, such promoting effect was not increased any more (FIG. 9).

On the other hand, a Rad51 protein showing sequence similarity to Rad51B inhibited the catenation of DNA molecules by the Ev1 protein, regardless of the presence or absence of ATP (FIG. 8b). These results suggest that the activity of the Ev1 protein should be promoted by specific functional interaction between the Ev1 and Rad51B proteins.

### 7. EVH2 Domain of Ev1 Protein

In order to identify a functional domain that causes the catenation of single-stranded DNA molecules by the Ev1 protein, two Ev1 fragments comprising amino acid residues at positions 1-221 and at positions 222-418, namely, Ev1 (1-221) and Ev1 (222-418), were purified. Purification of Ev1 (1-221) and Ev1 (222-418) was carried out in accordance with the protocols used in purification of the entire-length Ev1 protein.

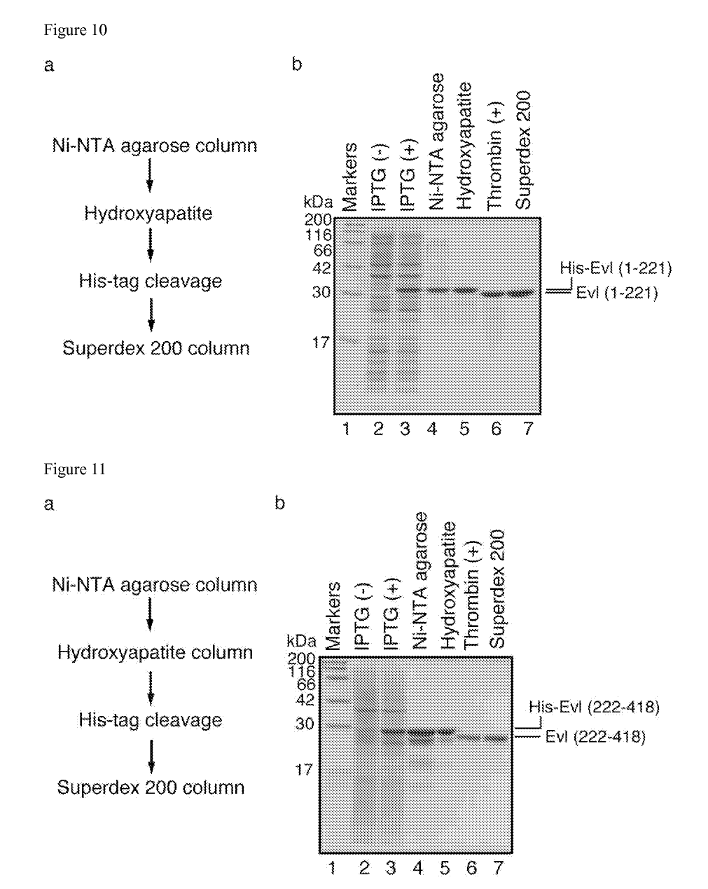

Constructs used in expression of Ev1 (1-221) and Ev1 (222-418) were produced according to an ordinary method. Thereafter, Escherichia coli (BL21(DE3)) was transformed with each construct, so as to allow it to express each protein. With regard to Ev1 (1-221), a cell extract was recovered, it was then passed through a Ni-NTA agarose column (Invitrogen), and the obtained peak fraction was then mixed with hydroxyapatite (Bio-Rad). After the mixture had been centrifuged, a supernatant that had not bound to the hydroxyapatite was recovered, and a His tag was removed by a thrombin treatment. Thereafter, Ev1 (1-221) was separated from the tag by Superdex200 (GE Healthcare) column chromatography. The Ev1 (1-221) obtained by Superdex200 column chromatography was used in the subsequent experiment (FIG. 10). On the other hand, with regard to Ev1 (222-418), a cell extract was recovered, and it was then passed through a Ni-NTA agarose column (Invitrogen). Thereafter, the obtained peak fraction was passed through hydroxyapatite (Bio-Rad), so that a peak fraction was recovered. A His tag was removed from the recovered Ev1 (222-418) by a thrombin treatment. Subsequently, the Ev1 (222-418) was separated from the tag by Superdex200 (GE Healthcare) column chromatography. The Ev1 (222-418) obtained by Superdex200 column chromatography was used in the subsequent experiment (FIG. 11).

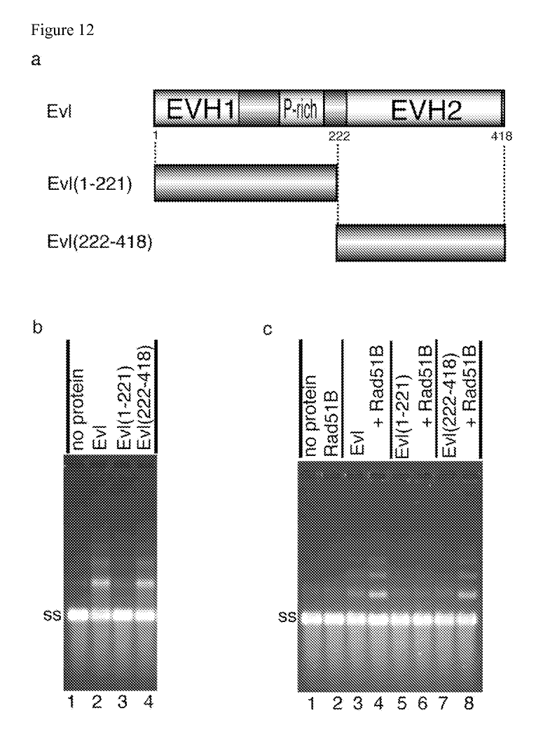

An Ev1 (1-221) mutant comprises EVH1 and a proline-rich domain, whereas an Ev1 (222-418) mutant comprises an EVH2 domain. As shown in FIG. 12b, the Ev1 (222-418) mutant exhibited ssDNA catenation activity (lane 4), but the Ev1 (1-221) mutant did not exhibit such ssDNA catenation activity (lane 3). In addition, the activity of the Ev1 (222-418) mutant was promoted by the Rad51B protein (FIG. 12c, lane 8). In contrast, in the case of the Ev1 (1-221) mutant, although the Rad51B protein was added thereto, no activity was detected (FIG. 12c, lane 6).

Accordingly, it is considered that the EVH2 domain is necessary for the ssDNA catenation activity of the Ev1 protein.

### 8. Effect of Promoting Catenation of Single-Stranded DNA Molecules Caused by Coexistence of DNA Topoisomerase Type I Protein and Ev1 Protein

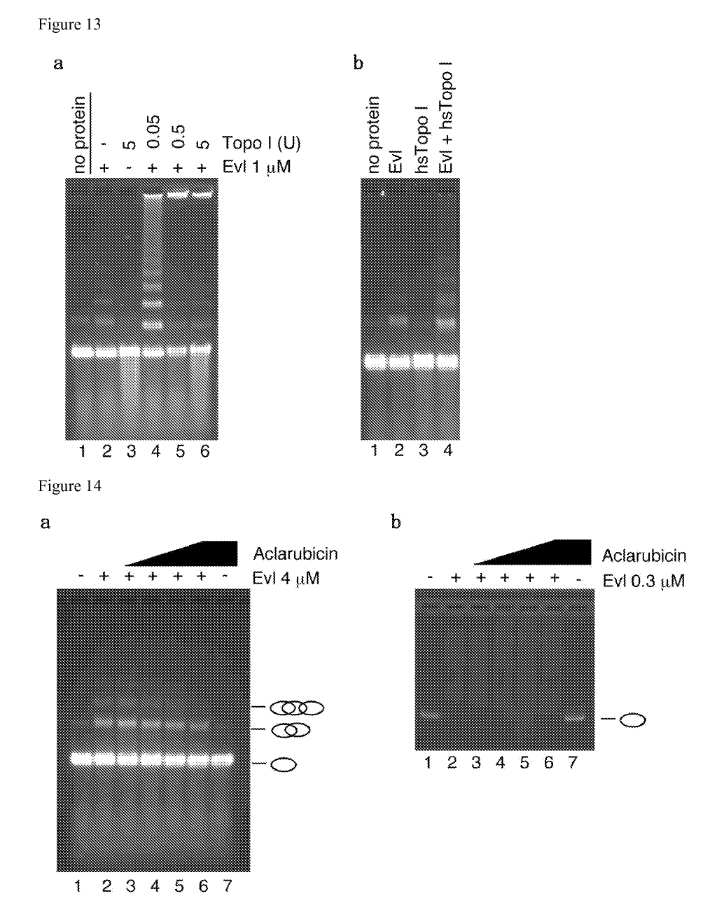

A 20 μM φ×174 single-stranded DNA, a 4 μM Ev1 protein, and a DNA topoisomerase type I protein in each concentration or unit number (derived from Escherichia coli or a human) were added to 10 μl of a reaction solution (20 mM HEPES, 1 mM DTT, 100 μg/ml BSA, and 1 mM MgCl₂). The obtained mixture was reacted at 37° C. for 1 hour. Thereafter, the sample was subjected to a deproteination treatment using 0.2% SDS and 1.3 mg/ml proteinase K. The obtained product was separated by 0.9% agarose gel electrophoresis, and a DNA band was then stained with SYBR Gold (Invitrogen) (FIG. 13).

As a result, it was revealed that the catenation of single-stranded DNA molecules is promoted in the coexistence of the Ev1 protein and the DNA topoisomerase type I protein derived from Escherichia coli (FIG. 13a, lanes 4-5) or derived from a human (FIG. 13b, lane 4).

### 9. Searching for Low Molecular Weight Compound that Affects Single-Stranded DNA Catenation Activity of Ev1 Protein

A low molecular weight compound that can be used to regulate at the correct time right the single-stranded DNA catenation activity of the Ev1 protein was searched. As a result, aclarubicin, dequalinium, DIDS, β-rubromycin, and 3-ATA were discovered as such compounds.

The inhibitory activity of each of the above compounds was detected as follows.

With regard to the influence of each compound on the catenation activity of the Ev1 protein (FIGS. 14a, 15a, 16, 17a, and 18a), each compound in final concentrations of 1, 5, 10, and 20 μM, a φ×174 single-stranded DNA in a final concentration of 20 μM, and an Ev1 protein in a final concentration of 4 μM were added to 10 μl of a reaction solution (20 mM HEPES, 1 mM DTT, 100 μg/ml BSA, and 1 mM MgCl₂). The obtained mixture was reacted at 37° C. for 1 hour. After completion of the reaction, 2 μl of a PK solution (0.2% SDS and 1.3 mg/ml proteinase K) was added to the reaction solution, and the obtained mixture was then reacted at 37° C. for 15 minutes. Thereafter, the reaction product was electrophoresed on a 0.8% HGT agarose gel, and DNA was then detected with SYBR Gold.

Moreover, in a gel shift method involving single-stranded DNA binding activity (FIGS. 14b, 15b, 16b, 17b, and 18b), each compound in final concentrations of 1, 5, 10, and 20 μM, a φ×174 single-stranded DNA in a final concentration of 20 μM, and an Ev1 protein in a final concentration of 0.3 μM were added to 20 μl of a reaction solution (20 mM HEPES, 1 mM DTT, 100 μg/ml BSA, and 1 mM MgCl₂). The obtained mixture was reacted at 37° C. for 15 minutes. Thereafter, the reaction product was electrophoresed on a 0.8% HGT agarose gel, and DNA was then detected with ethidium bromide.

### 9-1. Aclarubicin

Aclarubicin is an anthracycline antitumor agent, cardiotoxicity of which has been significantly decreased. It is also an agent for inhibiting the catalytic activity of topoisomerase I/II. Moreover, aclarubicin suppresses the chymotrypsin-like activity of 20S proteasome, so that it also suppresses the decomposition of a ubiquitinated protein. Such aclarubicin has been known to inhibit IL-1β-induced iNOS generation in aorticsmooth muscle cells. Aclarubicin is used on trial as an antitumor antibiotic (product name: Aclacinon) at clinical sites. This agent binds to the DNA of a cancer cell to strongly inhibit nucleic acid synthesis and RNA synthesis, and thus it is used for the purpose of alleviating and improving the subjective and objective symptoms of stomach cancer, lung cancer, breast cancer, malignant lymphoma, and acute leukemia. In the present invention, it was discovered that such aclarubicin inhibits the activity of an Ev1 protein to catenate single-stranded DNA molecules (FIG. 14a). Furthermore, the influence of aclarubicin on the activity of the Ev1 protein to bind to a single-stranded DNA was examined by a gel shift assay method (FIG. 14b). As a result, it was found that aclarubicin does not change the activity of the Ev1 protein to bind to circular single-stranded DNA. From the results of the aforementioned analysis, it became clear that aclarubicin inhibits the single-stranded DNA catenation activity of the Ev1 protein, without inhibiting the single-stranded DNA binding activity of the same protein.

### 9-2. Dequalinium

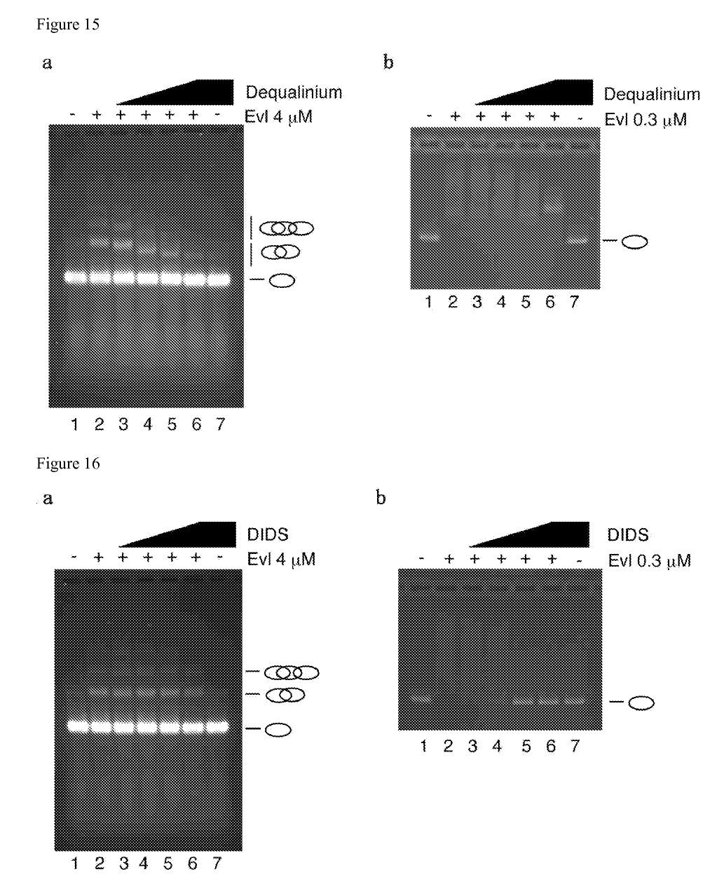

Dequalinium (dequalinium chloride) is an antitumoral, PKC-inhibitory agent. When UV is applied to dequalinium, such dequalinium covalently binds to PKCα or PKCβ so as to irreversibly inhibit them. Dequalinium is a strong, selective non-peptide blocker to an apamin-sensitive low transferable Ca²⁺-activated K⁺ channel, and it blocks neurotransmission. Moreover, it is accumulated selectively in the mitochondria of cancer cells, so that it inhibits energy production. Such dequalinium is used with a product name, “Nodoman Troche,” at clinical sites. Since this agent acts on the proteins of bacteria and kills bacteria existing in the mouth or throat, it is used for prevention of infection such a spharyngitis, tonsillitis, stomatitis, and oral wound including wound of tooth extraction. In the present invention, it was found that dequalinium inhibits the single-stranded DNA catenation activity of the Ev1 protein (FIG. 15a). Furthermore, the influence of dequalinium on the activity of the Ev1 protein to bind to a single-stranded DNA was examined by a gel shift assay method (FIG. 15b). As a result, it was found that dequalinium changes the binding manner of the Ev1 protein that binds to a single-stranded DNA. From the results of the aforementioned analysis, it is considered that dequalinium changes the binding manner of the Ev1 protein that binds to a single-stranded DNA, and that it inhibits the activity of the Ev1 protein to catenate a circular single-stranded DNA.

### 9-3. DIDS (Disodium Salts)

DIDS is an anion transport inhibitor that inhibits Cl incorporation into neuroblastoma cells, and it exhibits antiulcer action. In addition, DIDS is also known to inhibit ATP transport into an endoplasmic reticulum. In the present invention, it was found that DIDS inhibits the single-stranded DNA catenation activity of the Ev1 protein (FIG. 16a). Moreover, the influence of DIDS on the activity of the Ev1 protein to bind to a single-stranded DNA was examined by a gel shift assay method (FIG. 16b). As a result, it was found that DIDS inhibits the single-stranded DNA binding activity of the Ev1 protein. From the results of the aforementioned analysis, it is considered that DIDS inhibits the single-stranded DNA binding activity of the Ev1 protein, so as to inhibit the single-stranded DNA catenation activity of the Ev1 protein.

9-4. β-rubromycin

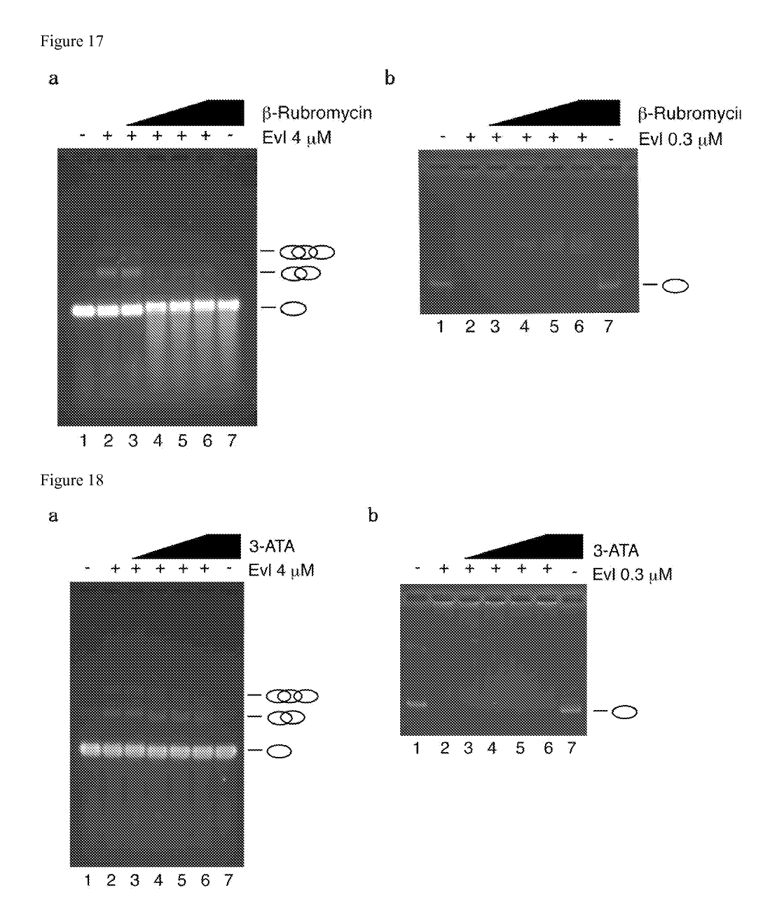

β-rubromycin is an inhibitory agent for HIV-I reverse transcriptase. In addition, βrubromycin is also known as an inhibitory agent for telomerase. In the present invention, it was found that β-rubromycin inhibits the single-stranded DNA catenation activity of the Ev1 protein (FIG. 17a). Moreover, the influence of β-rubromycin on the activity of the Ev1 protein to bind to a single-stranded DNA was examined by a gel shift assay method (FIG. 17b). As a result, it was found that β-rubromycin changes the binding manner of the Ev1 protein that binds to a single-stranded DNA. From the results of the aforementioned analysis, it is considered that β-rubromycin changes the binding manner of the Ev1 protein that binds to a single-stranded DNA, and that it inhibits the single-stranded DNA catenation activity of the Ev1 protein.

9-5. 3-ATA (3-amino-9-thio(10H)-acridone)

3-ATA is a CDK4 inhibitory agent. In addition, it is also found that 3-ATA suppresses the growth of p-16 mutation tumor. In the present invention, it was found that 3-ATA inhibits the single-stranded DNA catenation activity of the Ev1 protein (FIG. 18a). Moreover, the influence of 3-ATA on the activity of the Ev1 protein to bind to a single-stranded DNA was examined by a gel shift assay method (FIG. 18b). As a result, it was found that 3-ATA does not change the single-stranded DNA binding activity of the Ev1 protein. From the results of the aforementioned analysis, it was found that 3-ATA inhibits the single-stranded DNA catenation activity of the Ev1 protein, without inhibiting the single-stranded DNA binding activity of the Ev1 protein.

### INDUSTRIAL APPLICABILITY

Since the composition of the present invention has both activity of cleaving a single-stranded DNA and activity of binding the 5′-terminus of such single-stranded DNA to the 3′-terminus thereof, it can be used as an effective tool for manipulating a single-stranded DNA in a genetic engineering manner. Moreover, using the composition of the present invention, it also becomes possible to provide a single-stranded DNA molecular weight marker and the like. Thus, it can be anticipated that the composition of the present invention will greatly contribute to the progression of research and development in the biological and medical fields.

Sequence Listing

1. A composition for cleaving a single-stranded DNA and/or binding the 5′-terminus of such single-stranded DNA to the 3′-terminus thereof, which comprises a protein described in the following (a) or (b) and a DNA topoisomerase type I protein:
(a) a protein having the amino acid sequence shown in SEQ ID NO: 2, 4, 6, 8, 10, 12, or 20; or
(b) a protein having an amino acid sequence comprising a substitution, deletion, or insertion of one or several amino acids with respect to the amino acid sequence shown in SEQ ID NO: 2, 4, 6, 8, 10, 12, or 20.

2. The composition according to claim 1, wherein said composition further comprises Mg²⁺ or Ca²⁺.

3. The composition according to claim 2, wherein a concentration of said Mg²⁺ is between 0.5 mM and 2.0 mM.

4. The composition according to claim 1, wherein it said composition further comprises a Rad51B protein and ATP.

5. The composition according to claim 4, wherein the molar ratio of the protein described in (a) or (b) of claim 1 to said Rad51B is from 1:0.5 to 1:4.

6. The composition according to claim 4, wherein a concentration of said ATP is between 0.5 mM and 2.0 mM.

7. (canceled)

8. A composition for cleaving a single-stranded DNA and/or binding the 5′-terminus of such single-stranded DNA to the 3′-terminus thereof, which comprises a recombinant vector comprising a nucleic acid described in the following (a) or (b) and an expression vector of a DNA topoisomerase type I protein:
(a) a nucleic acid having the nucleotide sequence shown in SEQ ID NO: 1, 3, 5, 7, 9, 11, or 19; or
(b) a nucleic acid, which hybridizes under stringent conditions with a complementary strand of the nucleic acid having the nucleotide sequence shown in SEQ ID NO: 1, 3, 5, 7, 9, 11, or 19.

9. A composition for cleaving a single-stranded DNA and/or binding the 5′-terminus of such single-stranded DNA to the 3′-terminus thereof, which comprises a recombinant vector comprising a nucleic acid described in the following (a) or (b) and an expression vector of a DNA topoisomerase type I protein:
(a) a nucleic acid encoding a polypeptide having the amino acid sequence shown in SEQ ID NO: 2, 4, 6, 8, 10, 12, or 20; or
(b) a nucleic acid encoding a polypeptide, which has an amino acid sequence comprising a substitution, deletion, or insertion of one or several amino acids with respect to the amino acid sequence shown in SEQ ID NO: 2, 4, 6, 8, 10, 12, or 20.

10. A method for producing a single-stranded DNA marker by reacting the composition according claim 1 with a single-stranded DNA.

11. A single-stranded DNA marker produced by the method according to claim 10.

12. A composition for inhibiting Ev1 protein activity, which comprises one or multiple compounds selected from the compound group consisting of aclarubicin, dequalinium, DIDS, β-rubromycin, and 3-ATA.

13. A method for producing a single-stranded DNA marker by reacting the composition according to claim 8 with a single-stranded DNA.

14. A method for producing a single-stranded DNA marker by reacting the composition according to claim 9 with a single-stranded DNA.

15. A method comprising reacting at least one compound selected from the group consisting of aclarubicin, dequalinium, DIDS, β-rubromycin, and 3-ATA, with Ev1 protein so as to inhibit activity of said Ev1 protein.


SEQUENCE LISTING (20 sequences)

SEQ ID NO: 1 - DNA, 1257 bp, Homo sapiens
atggccacaagtgaacagagtatctgccaagcccgggcttccgtgatggtctacgatgacaccagtaagaaatgggtaccaatcaaacctggccagcagggattcagccggatcaacatctaccacaacactgccagcaacaccttcagagtcgttggagtcaagttgcaggatcagcaggttgtgatcaattattcaatcgtgaaagggctgaagtacaatcaggccacgccaaccttccaccagtggcgagatgcccgccaggtctacggcttaaactttgcaagtaaagaagaggcaaccacgttctccaatgcaatgctgtttgccctgaacatcatgaattcccaagaaggaggcccctccagccagcgtcaggtgcagaatggcccctctcctgatgagatggacatccagagaagacaagtgatggagcagcaccagcagcagcgtcaggaatctctagaaagaagaacctcggccacagggcccatcctcccaccaggacatccttcatctgcagccagcgcccccgtctcatgtagtgggcctccaccgccccccccacccccagtcccacctccacccactggggctaccccacctcccccacccccactgccagccggaggagcccaggggtccagccacgacgagagctccatgtcaggactggccgctgccatagctggggccaagctgagaagagtccaacggccagaagacgcatctggaggctccagtcccagtgggacctcaaagtccgatgccaaccgggcaagcagcgggggtggcggaggaggcctcatggaggaaatgaacaaactgctggccaagaggagaaaagcagcctcccagtcagacaagccagccgagaagaaggaagatgaaagccaaatggaagatcctagtacctccccctctccggggacccgagcagccagccagccacctaactcctcagaggctggccggaagccctgggagcggagcaactcggtggagaagcctgtgtcctcgattctgtccagaaccccgtctgtggcaaagagccccgaagctaagagcccccttcagtcgcagcctcactctaggatgaagcctgctgggagcgtgaatgacatggccctggatgccttcgacttggaccggatgaagcaggagatcctagaggaggtggtgagagagctccacaaggtgaaggaggagatcatcgacgccatcaggcaggagctgagtgggatcagcaccacgtaa

SEQ ID NO: 2 - protein, 418 aa, Homo sapiens
MATSEQSICQARASVMVYDDTSKKWVPIKPGQQGFSRINIYHNTASNTFRVVGVKLQDQQVVINYSIVKGLKYNQATPTFHQWRDARQVYGLNFASKEEATTFSNAMLFALNIMNSQEGGPSSQRQVQNGPSPDEMDIQRRQVMEQHQQQRQESLERRTSATGPILPPGHPSSAASAPVSCSGPPPPPPPPVPPPPTGATPPPPPPLPAGGAQGSSHDESSMSGLAAAIAGAKLRRVQRPEDASGGSSPSGTSKSDANRASSGGGGGGLMEEMNKLLAKRRKAASQSDKPAEKKEDESQMEDPSTSPSPGTRAASQPPNSSEAGRKPWERSNSVEKPVSSILSRTPSVAKSPEAKSPLQSQPHSRMKPAGSVNDMALDAFDLDRMKQEILEEVVRELHKVKEEIIDAIRQELSGISTT

SEQ ID NO: 3 - DNA, 1091 bp, Homo sapiens
tgcaggatcagcaggttgtgatcaattattcaatcgtgaaagggctgaagtacaatcaggccacgccaaccttccaccagtggcgagatgcccgccaggtctacggcttaaactttgcaagtaaagaagaggcaaccacattctccaatgcaatgctgtttgccctgaacatcatgaattcccaagaaggaggcccctccagccagcgtcaggtgcagaatggcccctctcctgatgagatggacatccagagaagacaagtgatggagcagcaccagcagcagcgtcaggaatctctagaaagaagaacctcggccacagggcccatcctcccaccaggacatccttcatctgcagccagcgcccccgtctcatgtagtgggcctccaccgccccccccacccccagtcccacctccacccactggggctaccccacctcccccacccccactgccagccggaggagcccaggggtccagccacgacgagagctccatgtcaggactggccgctgccatagctggggccaagctgagaagagtccaacggccagaagacgcatctggaggctccagtcccagtgggacctcaaagtccgatgccaaccgggcaagcagcgggggtggcggaggaggcctcatggaggaaatgaacaaactgctggccaagaggagaaaagcagcctcccagtcagacaagccagccgagaagaaggaagatgaaagccaaatggaagatcctagtacctccccctctccggggacccgagcagccagccagccacctaactcctcagaggctggccggaagccctgggagcggagcaactcggtggagaagcctgtgtcctcgattctgtccagaaccccgtctgtggcaaagagccccgaagctaagagcccccttcagtcgcagcctcactctaggatgaagcctgctgggagcgtgaatgacatggccctggatgccttcgacttggaccggatgaagcaggagatcctagaggaggtggtgagagagctccacaaggtgaaggaggagatcatcgacgccatcaggcaggagctgagtgggatcagcaccacgtaa

SEQ ID NO: 4 - protein, 362 aa, Homo sapiens
QDQQVVINYSIVKGLKYNQATPTFHQWRDARQVYGLNFASKEEATTFSNAMLFALNIMNSQEGGPSSQRQVQNGPSPDEMDIQRRQVMEQHQQQRQESLERRTSATGPILPPGHPSSAASAPVSCSGPPPPPPPPVPPPPTGATPPPPPPLPAGGAQGSSHDESSMSGLAAAIAGAKLRRVQRPEDASGGSSPSGTSKSDANRASSGGGGGGLMEEMNKLLAKRRKAASQSDKPAEKKEDESQMEDPSTSPSPGTRAASQPPNSSEAGRKPWERSNSVEKPVSSILSRTPSVAKSPEAKSPLQSQPHSRMKPAGSVNDMALDAFDLDRMKQEILEEVVRELHKVKEEIIDAIRQELSGISTT

SEQ ID NO: 5 - DNA, 1251 bp, Homo sapiens
atgagtgaacagagtatctgccaagcccgggcttccgtgatggtctacgatgacaccagtaagaaatgggtaccaatcaaacctggccagcagggattcagccggatcaacatctaccacaacactgccagcaacaccttcagagtcgttggagtcaagttgcaggatcagcaggttgtgatcaattattcaatcgtgaaagggctgaagtacaatcaggccacgccaaccttccaccagtggcgagatgcccgccaggtctacggcttaaactttgcaagtaaagaagaggcaaccacgttctccaatgcaatgctgtttgccctgaacatcatgaattcccaagaaggaggcccctccagccagcgtcaggtgcagaatggcccctctcctgatgagatggacatccagagaagacaagtgatggagcagcaccagcagcagcgtcaggaatctctagaaagaagaacctcggccacagggcccatcctcccaccaggacatccttcatctgcagccagcgcccccgtctcatgtagtgggcctccaccgccccccccacccccagtcccacctccacccactggggctaccccacctcccccacccccactgccagccggaggagcccaggggtccagccacgacgagagctccatgtcaggactggccgctgccatagctggggccaagctgagaagagtccaacggccagaagacgcatctggaggctccagtcccagtgggacctcaaagtccgatgccaaccgggcaagcagcgggggtggcggaggaggcctcatggaggaaatgaacaaactgctggccaagaggagaaaagcagcctcccagtcagacaagccagccgagaagaaggaagatgaaagccaaatggaagatcctagtacctccccctctccggggacccgagcagccagccagccacctaactcctcagaggctggccggaagccctgggagcggagcaactcggtggagaagcctgtgtcctcgattctgtccagaaccccgtctgtggcaaagagccccgaagctaagagcccccttcagtcgcagcctcactctaggatgaagcctgctgggagcgtgaatgacatggccctggatgccttcgacttggaccggatgaagcaggagatcctagaggaggtggtgagagagctccacaaggtgaaggaggagatcatcgacgccatcaggcaggagctgagtgggatcagcaccacgtaa

SEQ ID NO: 6 - protein, 416 aa, Homo sapiens
MSEQSICQARASVMVYDDTSKKWVPIKPGQQGFSRINIYHNTASNTFRVVGVKLQDQQVVINYSIVKGLKYNQATPTFHQWRDARQVYGLNFASKEEATTFSNAMLFALNIMNSQEGGPSSQRQVQNGPSPDEMDIQRRQVMEQHQQQRQESLERRTSATGPILPPGHPSSAASAPVSCSGPPPPPPPPVPPPPTGATPPPPPPLPAGGAQGSSHDESSMSGLAAAIAGAKLRRVQRPEDASGGSSPSGTSKSDANRASSGGGGGGLMEEMNKLLAKRRKAASQSDKPAEKKEDESQMEDPSTSPSPGTRAASQPPNSSEAGRKPWERSNSVEKPVSSILSRTPSVAKSPEAKSPLQSQPHSRMKPAGSVNDMALDAFDLDRMKQEILEEVVRELHKVKEEIIDAIRQELSGISTT

SEQ ID NO: 7 - DNA, 1218 bp, Mus musculus
atgagtgaacagagtatctgccaagcgcgggcctccgtgatggtctacgatgacaccagtaagaagtgggtaccgatcaagcctggccagcagggattcagccggatcaacatctaccacaacactgccagcagcaccttcagagtggtcggggtcaagctacaggaccagcaggttgtgatcaattattcaattgttaaagggctgaagtacaatcaggcaacacccaccttccatcagtggcgagatgcccgtcaggtctatggcttaaactttgcaagtaaggaagaagcaaccacattctccaatgccatgctctttgccctgaacatcatgaattcccaagaaggaggcccctccacacagcgtcaggtgcagaatggcccctctcctgaggagatggacatccagagaagacaagtaatggagcagcagcaccgccaggagtctctggagaggagaatctcggccacagggcccattctcccccctgggcatccctcatcggcagccagcaccactctctcctgtagtggacctccacccccgcctccacccccagttccacctccacccacagggtctactcccccacccccacccccactgccagctggaggagcccaggggaccaaccatgatgagagctctgcatcaggactggctgctgctctggcgggagccaagctaaggagggtgcagcggccagaagatgcatctggaggctccagtcctagtgggacttcaaagtccgatgccaaccgggcaagcagtgggggaggtggaggaggcctcatggaagaaatgaacaagctgctggctaagaggagaaaggcagcctcccagacagacaagcccgctgacagaaaggaagatgagagccaaacggaagaccctagcacctccccatccccaggtacccgagccaccagccagccacctaattcctcagaggctggcagaaaaccctgggaacggagcaactcggtggagaaacctgtgtcctcgttgctgtccagaaccccgtctgtggcaaagagccccgaagctaagagcccccttcagtcgcagcctcactctagggtgaagcctgctgggagtgtgaatgacgtgggcctggatgccttagatttggaccggatgaaacaggagatcctggaggaggtggttcgggagctgcacaaggtgaaggaggagatcattgatgccatcaggtag

SEQ ID NO: 8 - protein, 405 aa, Mus musculus
MSEQSICQARASVMVYDDTSKKWVPIKPGQQGFSRINIYHNTASSTFRVVGVKLQDQQVVINYSIVKGLKYNQATPTFHQWRDARQVYGLNFASKEEATTFSNAMLFALNIMNSQEGGPSTQRQVQNGPSPEEMDIQRRQVMEQQHRQESLERRISATGPILPPGHPSSAASTTLSCSGPPPPPPPPVPPPPTGSTPPPPPPLPAGGAQGTNHDESSASGLAAALAGAKLRRVQRPEDASGGSSPSGTSKSDANRASSGGGGGGLMEEMNKLLAKRRKAASQTDKPADRKEDESQTEDPSTSPSPGTRATSQPPNSSEAGRKPWERSNSVEKPVSSLLSRTPSVAKSPEAKSPLQSQPHSRVKPAGSVNDVGLDALDLDRMKQEILEEVVRELHKVKEEIIDAIR

SEQ ID NO: 9 - DNA, 1182 bp, Rattus norvegicus
atgagcgaacagagtatctgccaagcacgggcctccgtgatggtctacgatgacaccagtaagaaatgggtaccaatcaagcctggccagcagggattcagccggatcaacatctaccacaacactgccagcaacactttcagggttgtaggggtcaagctacaggatcagcaggttgtgatcaattattcaattgtgaaagggctgaagtacaatcaggcaacacccaccttccatcagtggcgagacgcccgtcaggtctatggcttaaactttgcgagtaagggagaagcaaccacattctccaacgcgatgctctttgccctgaacatcatgaactcccaagaaggaggcccctccacacagcgtcaggtgcagaatggcccctctcctgaggagatggacatccagagaagacaagtaatggagcagcagcaccgccaggagtctctggagagaagaatctccgccacagggcccattctcccccctgggcatccgtcatcggcagccagcgccaccttctcctgtagtggacctccacctccacctccacctccagttccacctccacccacagggtctactcccccgcccccgcccccgctgcctgctggaggagcccaggggaccaaccacgatgagagctctgcatcaggactggctgctgctctggcaggagccaagctaaggagggtgcagcggccagaggatgcatctggaggctccagtcctagcgggacttcaaagtccgatgccaaccgggcaagcagtgggggaggaggaggaggcctcatggaagaaatgaacaagctgctggctaagaggagaaaggcagcctcccagacagacaagcccgctgacagaaaggaagatgagaaccaaacggaagatcctagcacctccccatccccagggagccgagccaccagccagccacctaattcctcagaggctggccgaaagccctgggaacggagcaactcggtggagaaacctgtgtcctcgttgctgtccagggtgaagcctgctgggagtgtgaatgacgtgggcctggatgccttagatttggaccggatgaaacaggagattctggaggaggtggtccgagagctccacaaggtgaaggaggagatcatagatgccatcaggcaggaactaagtgggatcagcaccacataa

SEQ ID NO: 10 - protein, 393 aa, Rattus norvegicus
MSEQSICQARASVMVYDDTSKKWVPIKPGQQGFSRINIYHNTASNTFRVVGVKLQDQQVVINYSIVKGLKYNQATPTFHQWRDARQVYGLNFASKGEATTFSNAMLFALNIMNSQEGGPSTQRQVQNGPSPEEMDIQRRQVMEQQHRQESLERRISATGPILPPGHPSSAASATFSCSGPPPPPPPPVPPPPTGSTPPPPPPLPAGGAQGTNHDESSASGLAAALAGAKLRRVQRPEDASGGSSPSGTSKSDANRASSGGGGGGLMEEMNKLLAKRRKAASQTDKPADRKEDENQTEDPSTSPSPGSRATSQPPNSSEAGRKPWERSNSVEKPVSSLLSRVKPAGSVNDVGLDALDLDRMKQEILEEVVRELHKVKEEIIDAIRQELSGISTT

SEQ ID NO: 11 - DNA, 1257 bp, Gallus gallus
atggcgggcattgaacagagtatttgccaagcccgggcttcagttatggtctatgacgataccagtaagaaatgggtgccaatcaaacctggacagcagggattcagcagaatcaacatatatcacaacacggccacaaacaccttcagggttgttggagttaaactgcaagatcaacaagtagtgattaattactcaattgtgaaaggactgaagtacaatcaagcaacacctacctttcatcaatggcgcgatgcacggcaagtctatggcttgaattttgcaagcaaagaagaggctactacgttctccaatgcaatgctgtttgctctgaatataatgaattcacaagatggaggtccagctgcccagcgccaggtccagaatgggccatctccagatgagatggaagcacaaaggagacaagtgatggagcagcagcaacagcgccaagaatctctggaaagaagaacttctaccacaggcccagctctcccacccggccatcccagcggtgcttcagtgatccctgcttcatccgggggccccccacctccgccaccccccccggcccctccgccccccatgggagccgctcccccaccaccacccccgctgccagccggctccggccaaggggctgccagtgaagatgggtcggtgtcagggctcgcggctgctctggctggtgccaaactgaggagagttcagcggccagaagatggttcaggagggtccagccccagcggggtctctaagagcgatgccaatcgaacaagtagtggaggaggcagcggaggactaatggaagaaatgaataaattactggcaaaaaggaggaaagcagcgtcgcagtcagacaagccaggtgacaaaaaggaagaggaaagccaaaatgatgatgctagcacctctccttcaaccagtacacggggacccacccagcagcagcaaaattcatcagactctgggaagaagccatgggaaaggagcaattctgttgaaaagcctgtatcttcattactgtctagaaatccatccatggtgaagagctgtgaagctaagagccccacacaatcccacgtgtcttctaggatgaagccagtaagcagcagcaatgatgtggctatggatgccttagattttgatcggatgaaacaggaaatattggaggaagttgtaagagagttacacaaagtgaaagaggagataattgatgccatacggcaggagttgagtaggatcagtacaacatga

SEQ ID NO: 12 - protein, 418 aa, Gallus gallus
MAGIEQSICQARASVMVYDDTSKKWVPIKPGQQGFSRINIYHNTATNTFRVVGVKLQDQQVVINYSIVKGLKYNQATPTFHQWRDARQVYGLNFASKEEATTFSNAMLFALNIMNSQDGGPAAQRQVQNGPSPDEMEAQRRQVMEQQQQRQESLERRTSTTGPALPPGHPSGASVIPASSGGPPPPPPPPAPPPPMGAAPPPPPPLPAGSGQGAASEDGSVSGLAAALAGAKLRRVQRPEDGSGGSSPSGVSKSDANRTSSGGGSGGLMEEMNKLLAKRRKAASQSDKPGDKKEEESQNDDASTSPSTSTRGPTQQQQNSSDSGKKPWERSNSVEKPVSSLLSRNPSMVKSCEAKSPTQSHVSSRMKPVSSSNDVAMDALDFDRMKQEILEEVVRELHKVKEEIIDAIRQELSRISTT

SEQ ID NO: 13 - DNA, 1053 bp, Homo sapiens
atgggtagcaagaaactaaaacgagtgggtttatcacaagagctgtgtgaccgtctgagtagacatcagatccttacctgtcaggactttttatgtctttccccactggagcttatgaaggtgactggtctgagttatcgaggtgtccatgaacttctatgtatggtcagcagggcctgtgccccaaagatgcaaacggcttatgggataaaagcacaaaggtctgctgatttctcaccagcattcttatctactaccctttctgctttggacgaagccctgcatggtggtgtggcttgtggatccctcacagagattacaggtccaccaggttgtggaaaaactcagttttgtataatgatgagcattttggctacattacccaccaacatgggaggattagaaggagctgtggtgtacattgacacagagtctgcatttagtgctgaaagactggttgaaatagcagaatcccgttttcccagatattttaacactgaagaaaagttacttttgacaagtagtaaagttcatctttatcgggaactcacctgtgatgaagttctacaaaggattgaatctttggaagaagaaattatctcaaaaggaattaaacttgtgattcttgactctgttgcttctgtggtcagaaaggagtttgatgcacaacttcaaggcaatctcaaagaaagaaacaagttcttggcaagagaggcatcctccttgaagtatttggctgaggagttttcaatcccagttatcttgacgaatcagattacaacccatctgagtggagccctggcttctcaggcagacctggtgtctccagctgatgatttgtccctgtctgaaggcacttctggatccagctgtgtgatagccgcactaggaaatacctggagtcacagtgtgaatacccggctgatcctccagtaccttgattcagagagaagacagattcttattgccaagtcccctctggctcccttcacctcatttgtctacaccatcaaggaggaaggcctggttcttcaagcctatggaaattcctag

SEQ ID NO: 14 - protein, 350 aa, Homo sapiens
MGSKKLKRVGLSQELCDRLSRHQILTCQDFLCLSPLELMKVTGLSYRGVHELLCMVSRACAPKMQTAYGIKAQRSADFSPAFLSTTLSALDEALHGGVACGSLTEITGPPGCGKTQFCIMMSILATLPTNMGGLEGAVVYIDTESAFSAERLVEIAESRFPRYFNTEEKLLLTSSKVHLYRELTCDEVLQRIESLEEEIISKGIKLVILDSVASVVRKEFDAQLQGNLKERNKFLAREASSLKYLAEEFSIPVILTNQITTHLSGALASQADLVSPADDLSLSEGTSGSSCVIAALGNTWSHSVNTRLILQYLDSERRQILIAKSPLAPFTSFVYTIKEEGLVLQAYGNS

SEQ ID NO: 15 - DNA, 1053 bp, Mus musculus
atgagcagcaagaaactaagacgagtgggtttatctccagagctgtgtgaccgtttaagcagataccagattgttaactgtcagcactttttaagtctctccccactagaacttatgaaagtgactggcctgagttacagaggtgtccacgagcttcttcatacagtaagcaaggcctgtgccccgcagatgcaaacggcttatgagttaaagacacgaaggtctgcacatctctcaccggcattcctgtctactaccctgtgcgccttggatgaagcattgcacggtggtgtgccttgtggatctctcacagagattacaggtccaccaggttgcggaaaaactcagttttgcataatgatgagtgtcttagctacattacctaccagcctgggaggattagaaggggctgtggtctacatcgacacagagtctgcatttactgctgagagactggttgagattgcggaatctcgttttccacaatattttaacactgaggaaaaattgcttctgaccagcagtagagttcatctttgccgagagctcacctgtgaggggcttctacaaaggcttgagtctttggaggaagagatcatttcgaaaggagttaagcttgtgattgttgactccattgcttctgtggtcagaaaggagtttgacccgaagcttcaaggcaacatcaaagaaaggaacaagttcttgggcaaaggagcgtccttactgaagtacctggcaggggagttttcaatcccagttatcttgacgaatcaaattacgacccatctgagtggagccctcccttctcaagcagacctggtgtctccagctgatgatttgtccctgtctgaaggcacttctggatccagctgtttggtagctgcactaggaaacacatggggtcactgtgtgaacacccggctgattctccagtaccttgattcagagagaaggcagattctcattgccaagtctcctctggctgccttcacctcctttgtctacaccatcaagggggaaggcctggttcttcaaggccacgaaagaccatag

SEQ ID NO: 16 - protein, 350 aa, Mus musculus
MSSKKLRRVGLSPELCDRLSRYQIVNCQHFLSLSPLELMKVTGLSYRGVHELLHTVSKACAPQMQTAYELKTRRSAHLSPAFLSTTLCALDEALHGGVPCGSLTEITGPPGCGKTQFCIMMSVLATLPTSLGGLEGAVVYIDTESAFTAERLVEIAESRFPQYFNTEEKLLLTSSRVHLCRELTCEGLLQRLESLEEEIISKGVKLVIVDSIASVVRKEFDPKLQGNIKERNKFLGKGASLLKYLAGEFSIPVILTNQITTHLSGALPSQADLVSPADDLSLSEGTSGSSCLVAALGNTWGHCVNTRLILQYLDSERRQILIAKSPLAAFTSFVYTIKGEGLVLQGHERP

SEQ ID NO: 17 - DNA, 1017 bp, Arabidopsis thaliana
atgacggaatttgaactaatggagctgttagatgttggaatgaaagagataagatcagcaatttcattcatcagtgaagctacttctccaccatgtcaatctgctcgatctttactggagaagaaggtcgaaaacgaacatttatcaggtcatcttcctacacatttgaaggggttagattataccttgtgtggtgggataccttttggtgttcttactgagttagttggtcctcctggtattggtaaatcacagttttgcatgaaacttgcgttatcagcttcgtttccagtagcttatggaggattagatggtcgtgtgatatacatagatgtggaatccaagtttagttcaagaagggtgatagagatgggactggaaagctttccggaagtgtttcatcttaaaggaatggcacaagagatggctggaagaatccttgttttgcgtccaacatctttagctaactttactgaaagtatacaagaactcaagaattcaattcttcaaaaccaagtaaagcttctagtgattgatagtatgacagctcttctttcaggcgaaaacaaaccaggagctcagagacaacctcagttgggttggcatatctctttcttaaaatcgcttgctgaattttcacggattcctatagtggtgactaatcaagttagatctcaaaaccgcgatgaaactagtcagtattctttccaagctaaagttaaagatgaattcaaagacaacacaaagacatatgattctcaccttgttgctgcattggggattaactgggctcatgctgtaaccatccgactggtccttgaagccaagtcaggtcagagaatcattaaggtggcaaaatctcctatgtcgcctcctttagccttcccgttccatataacatcagctgggatttcattgctgagcgacaacgggactgaactgaaaggtccaggaatcaacaccattcatgctcgagggcacagcgacatgataaattttcacggggactgctcgtag

SEQ ID NO: 18 - protein, 338 aa, Arabidopsis thaliana
MTEFELMELLDVGMKEIRSAISFISEATSPPCQSARSLLEKKVENEHLSGHLPTHLKGLDYTLCGGIPFGVLTELVGPPGIGKSQFCMKLALSASFPVAYGGLDGRVIYIDVESKFSSRRVIEMGLESFPEVFHLKGMAQEMAGRILVLRPTSLANFTESIQELKNSILQNQVKLLVIDSMTALLSGENKPGAQRQPQLGWHISFLKSLAEFSRIPIVVTNQVRSQNRDETSQYSFQAKVKDEFKDNTKTYDSHLVAALGINWAHAVTIRLVLEAKSGQRIIKVAKSPMSPPLAFPFHITSAGISLLSDNGTELKGPGINTIHARGHSDMINFHGDCS

SEQ ID NO: 19 - DNA, 594 bp, Homo sapiens
atgtcaggactggccgctgccatagctggggccaagctgagaagagtccaacggccagaagacgcatctggaggctccagtcccagtgggacctcaaagtccgatgccaaccgggcaagcagcgggggtggcggaggaggcctcatggaggaaatgaacaaactgctggccaagaggagaaaagcagcctcccagtcagacaagccagccgagaagaaggaagatgaaagccaaatggaagatcctagtacctccccctctccggggacccgagcagccagccagccacctaactcctcagaggctggccggaagccctgggagcggagcaactcggtggagaagcctgtgtcctcgattctgtccagaaccccgtctgtggcaaagagccccgaagctaagagcccccttcagtcgcagcctcactctaggatgaagcctgctgggagcgtgaatgacatggccctggatgccttcgacttggaccggatgaagcaggagatcctagaggaggtggtgagagagctccacaaggtgaaggaggagatcatcgacgccatcaggcaggagctgagtgggatcagcaccacgtaa

SEQ ID NO: 20 - protein, 197 aa, Homo sapiens
MSGLAAAIAGAKLRRVQRPEDASGGSSPSGTSKSDANRASSGGGGGGLMEEMNKLLAKRRKAASQSDKPAEKKEDESQMEDPSTSPSPGTRAASQPPNSSEAGRKPWERSNSVEKPVSSILSRTPSVAKSPEAKSPLQSQPHSRMKPAGSVNDMALDAFDLDRMKQEILEEVVRELHKVKEEIIDAIRQELSGISTT